(19) United States
(12) Patent Application Publication
(10) Pub. No.: US 2024/0292893 A1
(43) Pub. Date: Sep. 5, 2024

(54) LIQUID STORAGE ELEMENT, WICKING ELEMENT, COOLING ELEMENT, CONDENSATE ABSORBING ELEMENT, AND SUPPORTING ELEMENT

(71) Applicant: ZHEJIANG MAIBO POLYMER MATERIALS CO., LTD., Zhejiang (CN)

(72) Inventors: Liping WANG, Zhejiang (CN); Xingfu ZHOU, Zhejiang (CN); Ding SHEN, Zhejiang (CN)

(73) Assignee: ZHEJIANG MAIBO POLYMER MATERIALS CO., LTD., Zhejiang (CN)

(21) Appl. No.: 18/283,814

(22) PCT Filed: Sep. 22, 2020
(86) PCT No.: PCT/CN2020/116733

§ 371 Date: Sep. 25, 2023

(30) Foreign Application Priority Data
Dec. 16, 2019 (CN) 201911291779.6

(51) Int. Cl. A24F 40/44; A24F 40/10; A24F 40/485; D01F 8/14

(52) U.S. Cl. CPC A24F 40/44; A24F 40/10; A24F 40/485; D01F 8/14

(57) ABSTRACT

A liquid storage element, a wicking element, a cooling element, a condensate absorbing element, and a supporting element using in an aerosol emission device is disclosed. The aforementioned elements have a three-dimensional network structure formed by thermally bonding bicomponent filaments, the bicomponent filaments have a sheath and a core. The aforementioned elements having a three-dimensional network structure formed by thermally bonding the bicomponent filaments according to the present invention can be conveniently assembled in the aerosol emission device.

### TECHNICAL FIELD

The present application relates to a liquid storage element, a wicking element. a cooling element. a condensate absorbing element, and a supporting element, and more particularly to a liquid storage element for storing and releasing liquids in an aerosol emission device which used for vaporizing or atomizing the liquids, a wicking element for conducting liquids, a cooling element for cooling aerosols, a condensate absorbing element for absorbing condensates, and a supporting element for supporting flavor changing members.

### BACKGROUND

When using traditional tobaccos, it has an impact on health when inhaling harmful substances such as tar generated when burning tobaccos. In electronic cigarettes, the method of heat to vaporize or atomize the active ingredients is usually used to replace the method of burning traditional tobaccos. In a common method of atomizing an e-liquid by heating, the e-liquid is stored in an oil reservoir, and the atomized aerosol is emitted from the oil reservoir, this type of electronic cigarette is prone to leak the e-liquid. In some electronic cigarettes, cotton or non-woven fabrics are wound on glass fiber or ceramic tubes, and then the e-liquid is injected on the cotton or non-woven fabrics. Because the cotton and non-woven fabrics lack three-dimensional shape and strength, it is difficult to automatically assemble, and the cotton or non-woven fabrics are nonuniform after winding, so that the local density is high. the liquid storage capacity is small, the ability to release e-liquid at a later stage of application is poor, and the residual rate of liquid after using is high. Similar problems exist in aerosol emission devices that vaporize or atomize liquids.

such as electric mosquito-repellent incense, electric aromatherapy, drug atomizing inhalation devices, and the like.

In addition, in electronic cigarettes, electric mosquito-repellent incense, electric aromatherapy, and drug atomizing inhalation devices, etc., a common structure is to install an atomizing core, such as porous ceramics with pre-embedded heating wires, in the aerosol emission device. An airflow passes through the atomizing device while the atomizing core is heated, the liquid is atomized and carried out by the airflow. In order to relatively stable conduct the liquid in the liquid storage portion to the atomizing core and prevent the liquid from leaking, the surface of the atomizing core is usually covered with non-woven fabrics and fixed in the aerosol emission device. Because the non-woven fabrics are soft and lack strength and is easy to wrinkle, it is difficult to manufacture an aerosol emission device with stable quality, and liquid leakage is prone to occur under the condition that wrinkles are serious. The method of coating the non-woven fabrics on the surface of the atomizing core requires a lot of labor, which is difficult to automate, has high cost and poor efficiency.

In addition, the temperature of traditional cigarettes during burning is around 800° C. when the moisture in the tobacco is formed into an aerosol at such a high temperature, most of the moisture therein is evaporated and the aerosol is relatively dry, the temperature sensed by the user when inhales the aerosol is relatively low. The aerosol or gasoloid generated by heating the aerosol substrate without burning may contain high moisture and aerosol agents for vaporizing the aerosol substrate, such as propylene glycol, glycerin, etc., so that the temperature sensed by the user when inhales the aerosol is higher. The heat-not-burn aerosol that are not properly cooled can even cause the user's mouth to burn. Smokers experience the same problem when using heat-not-burn traditional Chinese medicine.

A cooling element may be employed at the downstream of the aerosol substrate to absorb heat from the aerosol to cool the aerosol. The aerosol conducts its own heat to the cooling element by heat exchange to reduce its temperature, the temperature of the cooling element rises after absorbs the heat of the aerosol, if the substance in the cooling element undergoes a phase transition process such as melting after absorbing heat, the heat of the aerosol can be more absorbed, so that the cooling effect to the aerosol is more significant. In order to make the heat exchange sufficiently. the cooling element needs to have a large surface area in contact with the aerosol. With reference to the widely used finned heat exchanger, the cooling element can be made from a sheet-shaped substance. CN104203015A discloses a method which a cooling element made from a sheet material is used to cool a heat-not-burn aerosol. But from the point of view of the heat exchange contact area, the sheet is a two-dimensional structure and has a small specific surface area. In addition, according to the disclosure of CN104203015A, the cooling element made from a sheet cannot be adjacent to an aerosol substrate, and the cooling element and the aerosol substrate need to be separated by other elements. Apparently, the cooling element made from the sheet also cannot efficiently absorb the small droplets in the aerosol. In conclusion, in aerosol emission devices, there are many limitations on that the cooling element is made from the sheet. Similar problems exist in aerosol emission devices such as drug atomizing inhalation devices, etc. that heat liquids to vaporize or atomize.

In addition, when using traditional tobaccos, it has an impact on health when inhaling harmful substances such as tar generated when burning tobaccos, and electronic atomized cigarettes employ heat an atomizing solvent to intake nicotine or nicotine salt, this method does not generate tar. The commonly used solvent in electronic atomized cigarettes is 1, 2 propylene glycol and glycerin. their boiling points are 188.2° C. and 290° C. respectively, and since the temperature of the peripheral wall of an aerosol channel is lower, the condensate is continuously increased during the process of the atomized aerosol passing through the aerosol channel. When a large amount of condensate enters the user's mouth, it will seriously affect the user's taste, therefore, most of the condensate is removed before the aerosol enters the user's mouth, so that the smoking experience of the electronic atomized cigarettes can be greatly improved. The condensate can be removed by contacting the condensate with a suitable absorbing material (a condensate absorbing element). In some atomization devices, the condensate will settle to the bottom of the atomizer, in this case, a condensate absorber can be installed at the bottom of the atomizer to prevent the condensate from penetrating into the host. The more common condensate absorbing element is stacked together by multiple layers of non-woven fabrics and die-cut to a desired size and shape, since the non-woven fabric is soft and lacks a fixed three-dimensional shape, which is difficult to be installed or fixed in a narrow electronic atomizing aerosol channel. Another common condensate absorbing element is fibers or wood pulp compressed into sheets, cutting into a desired size and shape as needed, or punching to form airflow channels as needed, this condensate absorbing element is commonly known as high-pressure cotton. The advantage is that it can be made into a three-dimensional shape and is easy to install, the disadvantage of the high-pressure cotton is that it expands significantly after absorbing the condensate, and an air resistance of the aerosol channel is unstable during use, which will affect the user's experience. Similar problems exist in the emission devices such as drug atomizing inhalation devices, etc. that vaporize or atomize liquids.

In addition, when using traditional tobaccos, it has an impact on health when inhaling harmful substances such as tar generated when burning tobaccos. The inhalation of nicotine or nicotine salts by atomizing is becoming more and more widely used because this method does not produce harmful substances such as tar. Similar atomization techniques can also be used to inhale substances such as drugs. In order to improve the taste, various flavors are usually added to an atomized liquid. But there are two problems. On the one hand, the flavors are easy to volatilize and will gradually lost during product storage, on the other hand, the flavors may be decomposed or produce harmful substances due to high temperature during heating and atomization, thus creating additional safety risks.

### SUMMARY

In order to solve the technical problems in the related art, the present invention provides a liquid storage element for storing and releasing liquids in an aerosol emission device, wherein the liquid storage element has a three-dimensional network structure formed by thermally bonding bicomponent filaments, and the bicomponent fibers have a sheath and a core.

The liquid storage element with the three-dimensional network structure which is formed by thermally bonding the bicomponent filaments can be easily assembled in the aerosol emission device. This liquid storage element has a lower density and a higher porosity, so that it can store more liquid per unit volume and release the liquid more efficiently. Since the liquid is stored in the capillary gap of the liquid storage element, it not easy to leak during the storage, transportation and application. The liquid storage element of the present invention can be used not only in electronic cigarettes, but also in electric mosquito-repellent incense, electric aromatherapy and drug atomization device which are having atomizers.

The present invention also provides a wicking element for conducting liquids in an aerosol emission device, wherein the wicking element has a three-dimensional network structure formed by thermally bonding bicomponent fibers, and the bicomponent fibers have a sheath and a core.

The wicking element made by bonding the bicomponent fibers has a higher strength and a higher toughness, and is not prone to be wrinkled or broken during installation, so that it can be conveniently assembled in the aerosol emission device, and is easy to achieve automated assembly, improves efficiency, and saves the cost, and is especially suitable for manufacturing large-scale consumer products, such as electronic cigarettes and the like. Due to the fact that the bicomponent fibers are bonded to form the three-dimensional network structure, a large number of capillary pores communicating with each other are formed in the wicking element, these capillary pores facilitate rapid and stable conduction of the liquid therein and improve the stability of supplementing the liquid to the atomizing core, thereby improving the stability of the atomization. It can control the sizes of the capillary pores and capillary forces by selecting the fiber fineness and setting the density of the wicking element so that the wicking element is suitable for the requirements of different aerosol emission devices.

The wicking element of the present invention can be applied to the atomization of e-liquids of various electronic cigarettes, and can also be applied to the atomization of the liquids of electric mosquito-repellent incense and air freshener.

The present invention also provides a cooling element for cooling an aerosol generated in an aerosol emission device, wherein the cooling element has a three-dimensional network structure formed by thermally bonding bicomponent fibers, and the bicomponent fibers have a sheath and a core.

The cooling element made by bonding the bicomponent fibers has a large number of capillary pores, which has a good absorption effect on the condensate generated during cooling the aerosol, so that the aerosol becomes dry, which is beneficial to users to sense a lower temperature. The cooling element made by bonding the bicomponent fibers can be made into a hollow structure and a non-hollow structure, which can be used alone or in combination according to needs, so as to achieve an appropriate cooling effect and air resistance.

The cooling element made by bonding the bicomponent fibers has a large specific surface area, which is beneficial to improve the heat exchange efficiency with the aerosol. The core of the bicomponent fibers has a higher melting point than the sheath by 25° C. or more, and when the aerosol temperature is higher than the melting point of the sheath, the sheath is partially melted by contacting the high-temperature aerosol and absorbs a large amount of heat, so that the temperature of the aerosol is dropped rapidly. The high-melting point of the core of the bicomponent fibers acts as a skeleton, and the molten sheath turns into a viscous flow state and adheres to the core, thereby maintaining the integrity of the cooling element.

The cooling element made by bonding the bicomponent fibers can be made to have different porosities according to the requirements, so that the cooling element has a required radial hardness and axial rigidity, and it is convenient to be assembled with other elements such as an aerosol substrate into an aerosol emission device and is easy to achieve efficient automated assembly.

The cooling element of the present invention can be applied to various aerosol emission devices, such as the aerosol emission devices which containing essence, nicotine, caffeine, theophylline, vaporizable Chinese herbal medicinal component, and the like.

The present invention also provides a condensate absorbing element for absorbing condensate in an aerosol emission device, wherein the condensate absorbing element has a three-dimensional network structure formed by thermally bonding bicomponent filaments, and the bicomponent fibers have a sheath and a core.

The condensate absorbing element with a three-dimensional network structure which is formed by thermally bonding the bicomponent filaments, can be customized according to the structure of the aerosol emission device, so as to be conveniently assembled in a precise aerosol emission device. The preparing process can be controlled so that the condensate absorbing element has a greater rigidity in the axial direction than in the radial direction, which facilitates the condensate absorbing element to assemble in the aerosol emission device by the axial force, thereby improving the assembly efficiency, meanwhile the condensate absorbing element can be conveniently fixed in the aerosol emission device by utilizing its radial self-adaptive deformation.

The condensate absorbing element of the present invention is made by bonding the bicomponent fibers, has a three-dimensional network structure, can quickly absorb the condensate around the aerosol when contacting the aerosol and conduct it to various portions of the condensate absorbing element, which has high removal efficiency for the condensate in the aerosol and good user experience. The condensate absorbing element of the present invention has a lower density, a higher porosity, and a large absorption capacity per unit volume, that is suitable for the aerosol emission device with compact space

The condensate absorbing element of the present invention with a three-dimensional network structure formed by bonding the bicomponent fibers, does not expand or deform after absorbing the condensate, so that the aerosol channel has a stable airflow resistance, which is beneficial to maintain the stability of the air resistance in the process of using the aerosol emission device and improves user experience.

The sheath of the bicomponent fibers used to make the condensate absorbing element of the present invention may be polylactic acid, and the polylactic acid is a biodegradable material, which can reduce the environmental pollution caused by discarding the condensate absorbing element. Especially when the core of the bicomponent fibers also is polylactic acid, the discarded condensate absorbing element can be completely degraded by microorganisms in nature to generate carbon dioxide and water.

The present invention also provides a supporting element for supporting a flavor changing member in an aerosol emission device, wherein the supporting element has a three-dimensional network structure formed by thermally bonding bicomponent filaments, and the bicomponent fibers have a sheath and a core.

The supporting element with a three-dimensional network structure which is formed by thermally bonding the bicomponent fibers can be conveniently assembled in the aerosol emission device. The supporting element of the present invention can not only be used for electronic cigarettes, but also for drug atomizing devices, and the supporting element can also be used in a separate suction nozzle that is used in conjunction with the aerosol emission device.

In order to make the above-mentioned content of the present invention more obvious and easier to understand, preferred embodiments are hereinafter described in detail with reference to the accompanying drawings.

### BRIEF DESCRIPTION OF THE DRAWINGS

One or more embodiments are illustrated by way of example with reference to the pictures in the corresponding drawings, which do not constitute a limitation on the embodiments, elements having the same reference numerals in the accompanying drawings are represented as similar elements, unless specifically stated, the figures in the drawings do not constitute a proportion limitation.

FIG. 1a shows a longitudinal section view of a liquid storage element according to the first embodiment disclosed in the present invention.

FIG. 1b shows a cross-sectional view of the liquid storage element according to the first embodiment disclosed in the present invention.

FIG. 1c shows an enlarged cross-sectional schematic view of bicomponent fibers of FIGS. 1a and 1b.

FIG. 1d shows another enlarged cross-sectional schematic view of the bicomponent fibers of FIGS. 1a and 1b.

FIG. 2a shows a longitudinal section view of a liquid storage element according to the second embodiment disclosed in the present invention.

FIG. 2b shows a cross-sectional view of the liquid storage element according to the second embodiment disclosed in the present invention.

FIG. 3a shows a longitudinal section view of a liquid storage element according to the third embodiment disclosed in the present invention.

FIG. 3b shows a cross-sectional view of the liquid storage element according to the third embodiment disclosed in the present invention.

FIG. 4a shows a longitudinal section view of a liquid storage element according to the fourth embodiment disclosed in the present invention.

FIG. 4b shows a cross-sectional view of the liquid storage element according to the fourth embodiment disclosed in the present invention.

FIG. 5a shows a longitudinal section view of a liquid storage element according to the fifth embodiment disclosed in the present invention.

FIG. 5b shows a cross-sectional view of the liquid storage element according to the fifth embodiment disclosed in the present invention.

FIG. 6a shows a longitudinal section view of a liquid storage element according to the sixth embodiment disclosed in the present invention.

FIG. 6b shows a cross-sectional view of the liquid storage element according to the sixth embodiment disclosed in the present invention.

FIG. 7a shows a longitudinal section view of a liquid storage element according to the seventh embodiment disclosed in the present invention.

FIG. 7b shows a cross-sectional view of the liquid storage element according to the seventh embodiment disclosed in the present invention.

FIG. 8a shows a longitudinal section view of a wicking element according to the eighth embodiment disclosed in the present invention.

FIG. 8b shows a cross-sectional view of the wicking element according to the eighth embodiment disclosed in the present invention.

FIG. 8c shows an enlarged cross-sectional schematic view of bicomponent fibers of FIGS. 8a and 8b.

FIG. 8d shows another enlarged cross-sectional schematic view of the bicomponent fibers of FIGS. 8a and 8b.

FIG. 9a shows a longitudinal section view of a wicking element according to the ninth

embodiment disclosed in the present invention.

FIG. 9b shows a cross-sectional view of the wicking element according to the ninth embodiment disclosed in the present invention when the wicking element is cylinder.

FIG. 9c shows a cross-sectional view of the wicking element according to the ninth embodiment disclosed in the present invention when the wicking element is cuboid.

FIG. 9d shows a cross-sectional view of the wicking element according to the ninth embodiment disclosed in the present invention when the wicking element is elliptic cylinder.

FIG. 10a shows a longitudinal section view of a wicking element according to the tenth embodiment disclosed in the present invention.

FIG. 10b shows a cross-sectional view of the wicking element according to the tenth embodiment disclosed in the present invention when the wicking element is cylinder.

FIG. 10c shows a cross-sectional view of the wicking element according to the tenth embodiment disclosed in the present invention when the wicking element is cuboid.

FIG. 10d shows a cross-sectional view of the wicking element according to the tenth embodiment disclosed in the present invention when the wicking element is elliptic cylinder.

FIG. 11a shows a longitudinal section view of a cooling element according to the eleventh embodiment of the present invention.

FIG. 11b shows a cross-sectional view of the cooling element according to the eleventh embodiment of the present invention.

FIG. 11c shows an enlarged cross-sectional schematic view of bicomponent fibers of FIGS. 11a and 11b.

FIG. 11d shows another enlarged cross-sectional schematic view of the bicomponent fibers of FIGS. 11a and 11b.

FIG. 11e shows another cross-sectional view of the cooling element according to the eleventh embodiment of the present invention.

FIG. 12a shows a longitudinal section view of a cooling element according to the twelfth embodiment of the present invention.

FIG. 12b shows a cross-sectional view of the cooling element according to the twelfth embodiment of the present invention.

FIG. 13a shows a longitudinal section view of a cooling element according to the thirteenth embodiment of the present invention.

FIG. 13b shows a cross-sectional view of a high-temperature cooling section of the cooling element according to the thirteenth embodiment of the present invention.

FIG. 13c shows another cross-sectional view of the high-temperature cooling section of the cooling element according to the thirteenth embodiment of the present invention.

FIG. 13d shows a cross-sectional view of a low-temperature cooling section of the cooling element according to the thirteenth embodiment of the present invention.

FIG. 14a shows a longitudinal section view of a cooling element according to the fourteenth embodiment of the present invention.

FIG. 14b shows a cross-sectional view of a high-temperature cooling section of the cooling element according to the fourteenth embodiment of the present invention.

FIG. 14c shows a cross-sectional view of a low-temperature cooling section of the cooling element according to the fourteenth embodiment of the present invention.

FIG. 14d shows another cross-sectional view of the low-temperature cooling section of the cooling element according to the thirteenth embodiment of the present invention.

FIG. 15a shows a longitudinal section view of a cooling element according to the fifteenth embodiment of the present invention.

FIG. 15b shows a cross-sectional view of a high-temperature cooling section of the cooling element according to the fifteenth embodiment of the present invention.

FIG. 15c shows a cross-sectional view of a low-temperature cooling section of the cooling element according to the fifteenth embodiment of the present invention.

FIG. 16a shows a longitudinal section view of a cooling element according to the sixteenth embodiment of the present invention.

FIG. 16b shows a cross-sectional view of a high-temperature cooling section of the cooling element according to the sixteenth embodiment of the present invention.

FIG. 16c shows a cross-sectional view of a low-temperature cooling section of the cooling element according to the sixteenth embodiment of the present invention.

FIG. 17a shows a longitudinal section view of a cooling element according to the seventeenth embodiment of the present invention.

FIG. 17b shows a cross-sectional view of a high-temperature cooling section of the cooling element according to the seventeenth embodiment of the present invention. FIG. 17c shows a cross-sectional view of a low-temperature cooling section of the cooling element according to the seventeenth embodiment of the present invention.

FIG. 18a shows a longitudinal section view of a cooling element according to the eighteenth embodiment of the present invention.

FIG. 18b shows a cross-sectional view of the cooling element according to the eighteenth embodiment of the present invention.

FIG. 19a shows a longitudinal section view of a condensate absorbing element according to the nineteenth embodiment disclosed in the present invention.

FIG. 19b shows a cross-sectional view of the condensate absorbing element according to the nineteenth embodiment of the present invention.

FIG. 19c shows an enlarged cross-sectional schematic view of bicomponent fibers of FIGS. 19a and 19b.

FIG. 19d shows another enlarged cross-sectional schematic view of the bicomponent fibers of FIGS. 19a and 19b.

FIG. 20a shows a longitudinal section view of a condensate absorbing element according to the twentieth embodiment disclosed in the present invention.

FIG. 20b shows a cross-sectional view of the condensate absorbing element according to the twentieth embodiment disclosed in the present invention.

FIG. 21a shows a longitudinal section view of a condensate absorbing element according to the twenty-first embodiment disclosed in the present invention.

FIG. 21b shows a cross-sectional view of the condensate absorbing element according to the twenty-first embodiment disclosed in the present invention.

FIG. 22a shows a longitudinal section view of a condensate absorbing element according to the twenty-second embodiment disclosed in the present invention.

FIG. 22b shows a cross-sectional view of the condensate absorbing element according to the twenty-second embodiment disclosed in the present invention.

FIG. 23a shows a longitudinal section view of a condensate absorbing element according to the twenty-third embodiment disclosed in the present invention before installation.

FIG. 23b shows a cross-sectional view of the condensate absorbing element according to the twenty-third embodiment disclosed in the present invention.

FIG. 23c shows a longitudinal section view of the condensate absorbing element according to the twenty-third embodiment disclosed in the present invention after installation.

FIG. 24a shows a longitudinal section view of a condensate absorbing element according to the twenty-fourth embodiment disclosed in the present invention.

FIG. 24b shows a cross-sectional view of the condensate absorbing element according to the twenty-fourth embodiment disclosed in the present invention.

FIG. 25a shows a longitudinal section view of a supporting element according to the twenty-fifth embodiment disclosed in the present invention.

FIG. 25b shows a cross-sectional view of the supporting element according to the twenty-fifth embodiment disclosed in the present invention.

FIG. 25c shows an enlarged cross-sectional schematic view of bicomponent fibers of FIGS. 25a and 25b.

FIG. 25d shows another enlarged cross-sectional schematic view of the bicomponent fibers of FIGS. 25a and 25b.

FIG. 26a shows a longitudinal section view of a supporting element according to the twenty-sixth embodiment disclosed in the present invention.

FIG. 26b shows a cross-sectional view of the supporting element according to the twenty-sixth embodiment disclosed in the present invention.

FIG. 27a shows a longitudinal section view of a supporting element according to the twenty-seventh embodiment disclosed in the present invention.

FIG. 27b shows a cross-sectional view of the supporting element according to the twenty-seventh embodiment disclosed in the present invention.

### DETAILED DESCRIPTION

The embodiments of the present invention are described below by way of specific embodiments, and those skilled in the art can readily understand other advantages and functions of the present invention from the disclosure of the present invention.

Exemplary embodiments of the present invention will now be described with reference to the accompanying drawings; however, the invention may be embodied in many different forms and is not limited to the embodiments described herein, which are provided for the purpose of providing a detailed and complete disclosure of the present invention, and fully conveying the scope of the invention to those skilled in the art. The terminology shown in the exemplary embodiments in the drawings is not intended to be limiting of the present invention. In the drawings, the same elements/components generally use the same or similar reference numerals.

A poly-L-lactic acid of the present invention. PLLA for short, refers to polylactic acid made from monomer L-lactic acid, but in which a small amount of D-lactic acid may be randomly copolymerized, and having a melting point of 145° C. to 180° C.

A poly-D-lactic acid of the present invention. PDLA for short, refers to polylactic acid made from monomer D-lactic acid, but in which a small amount of L-lactic acid may be randomly copolymerized. and having a melting point of 145° C. to 180° C.

A poly-D, L-lactic acid of the present invention. PDLLA for short, refers to polylactic acid made from monomer D-lactic acid and monomer L-lactic acid and having a melting point of less than 145° C., and comprises amorphous PDLLA which doesn't have a melting point.

The melting point of the present invention was measured according to ASTM D3418-2015.

The term “phenol” refers to a class of compounds consisting of a hydroxyl group directly bonded to an aromatic group. Phenols include phenol, catechol, o-phenol, m-cresol, p-cresol, and the like.

As used herein, the terms including scientific and technical terms have the meanings commonly understood to one skilled in the art, unless otherwise indicated. In addition, it is to be understood that a term defined in commonly used dictionaries should be understood to have a consistent meaning in the context of its associated domain and should not be interpreted as an idealized or overly formal meaning.

### The First Embodiment

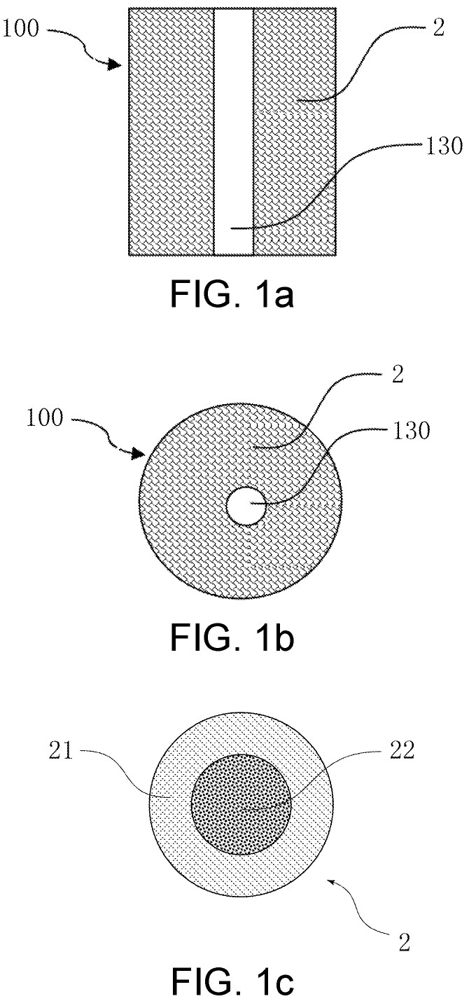

FIG. 1a shows a longitudinal section view of a liquid storage element according to the first embodiment disclosed in the present invention; FIG. 1b shows a cross-sectional view of the liquid storage element according to the first embodiment disclosed in the present invention.

As shown in FIGS. 1a and 1b, the liquid storage element according to the first embodiment of the present invention that is used for storing and releasing liquids in an aerosol emission device. A liquid storage element 100 has a three-dimensional network structure formed by thermally bonding bicomponent filaments 2, and the bicomponent fibers 2 have a sheath 21 and a core 22.

### <Shape of Liquid Storage Element>

The liquid storage element 100 may has a liquid storage element through-hole 130 axially penetrating therethrough. The liquid storage element through-hole 130 may be used as an aerosol channel in an aerosol emission device.

The liquid storage element 100 of the present embodiment may be made into an appropriate geometry according to the inner space of the aerosol emission device, for example. made into a cylindrical shape liquid storage element 100 suitable for a cylindrical aerosol emission device; a square column shape liquid storage element 100 suitable for a flat aerosol emission device; an elliptical column shape liquid storage element 100 suitable for an elliptical column shape aerosol emission device, etc.

The liquid storage element 100 has a liquid storage element through-hole 130 axially penetrating therethrough. An aerosol tube, such as a metal tube, a glass fiber tube, or a plastic tube. can be inserted into the liquid storage element through-hole 130, and an aerosol can be emitted from the aerosol tube. By providing the aerosol tube. the liquid storage element 100 can be better fixed, and can prevent the liquid from leaking from an atomizer (not shown) when the liquid storage element 100 is filled with liquids.

The portion that the liquid storage element 100 contacts the atomizer can be compressed to be a higher density portion, so that the liquid is enriched to the higher density portion during releasing, thereby improving the uniformity of the liquid release and further reducing the liquid residue after application.

### <Density of Liquid Storage Element>

The density of the liquid storage element 100 of the present embodiment is 0.03 to 0.25 g/cm³, such as 0.03 g/cm³, 0.04 g/cm³, 0.050 g/cm³, 0.055 g/cm³, 0.065 g/cm³, 0.08 g/cm³, 0.10 g/cm³, 0.12 g/cm³, 0.15 g/cm³, 0.18 g/cm³, 0.21 g/cm³, 0.25 g/cm³, preferably 0.04 to 0.12 g/cm³. When the density is less than 0.03 g/cm³, the liquid storage element 100 is difficult to manufacture. and the strength of the liquid storage element 100 is insufficient, which is not prone to be assembled in the aerosol emission device; when the density is 0.03 to 0.04 g/cm³, the strength of the liquid storage element 100 axially provided with a channel is slightly insufficient, which is not easy to be assembled; when the density is greater than 0.15 g/cm³, the liquid release efficiency of the liquid storage element 100 at a later stage of application is slightly poor, and the liquid residue after application is high; when the density is greater than 0.25 g/cm³, the liquid storage capacity of the liquid storage element 100 per unit volume is too small, and the liquid release efficiency of the liquid storage element 100 at the later stage of application is poor, the liquid residue after application is high, which is not beneficial to use in the aerosol emission device with a narrow space.

In the range of 0.04 to 0.12 g/cm³, according to the viscosity, surface tension and application requirements of the stored liquid, an appropriate density is selected, so that the liquid storage element 100 not only has a sufficient capillary force to prevent liquid leakage, but also good release properties, and the liquid storage capacity of the liquid storage element 100 can be maximized, which is beneficial to manufacture a compact aerosol emission device. It should be noted that, in order to prevent leakage during storage, transportation and application, the volume of liquid loaded into the liquid storage element 100 preferably does not exceed 90% of the capillary void volume in the liquid storage element 100.

In order to more intuitively illustrate the relationship between the density of the liquid storage element 100 and the using effect, in the present embodiment, the liquid storage elements 100 with different densities are manufactured, and the corresponding aerosol emission devices are assembled for suction testing. The atomizing core is a glass fiber wrapped with a heating wire. The liquid storage element 100 is made by thermally bonding the bicomponent staple fibers having 3-denier, the sheath 21 is polyethylene, the core 22 is polypropylene, the height of the liquid storage element 100 is 29 mm, and the volume is 1.91 cm³. The density of the liquid storage elements 100 is 0.04 g/cm³, 0.055 g/cm³, 0.08 g/cm³, 0.12 g/cm³, 0.15 g/cm³, and 0.20 g/cm³, respectively, the atomized liquid is a mixture of propylene glycol and glycerin, and the liquid injection amount is 1.62 g. A smoking machine is used for testing. The test conditions are as follows: a puff for 3 seconds duration and a puff interval for 27 seconds, take 2 puffs per minute, a puff volume of 55 ml, collect the atomization amount of each puff of 50 puffs, and repeat the test 20 times for each product, the design capacity of the lithium battery is 400 puffs (the actual test is that when 405-436 puffs the battery is exhausted). The data is calculated to obtain an average value (unit mg), a co-efficient of variation (CV for short) of atomization amount of each puff, and a liquid residual rate and a CV after smoking 400 puffs. The results are as follows:

| | Puff | | | | | | | | | residual |
|---|---|---|---|---|---|---|---|---|---|---|
| Density | Numbers | 1-50 | 51-100 | 101-150 | 151-200 | 201-250 | 251-300 | 301-350 | 351-400 | rate |
| 0.04 | Atomization | 3.55 | 3.59 | 3.62 | 3.45 | 3.52 | 3.41 | 3.36 | 2.57 | 16.5% |
| | Amount | | | | | | | | | |
| | CV | 5.3% | 2.1% | 1.1% | 6.7% | 3.4% | 5.3% | 9.8% | 43.6% | 6.5% |
| 0.055 | Atomization | 3.44 | 3.62 | 3.61 | 3.39 | 3.57 | 3.38 | 3.28 | 2.50 | 17.4% |
| | Amount | | | | | | | | | |
| | CV | 1.2% | 1.3% | 2.9% | 7.5% | 1.1% | 4.0% | 11.2% | 48.4% | 7.3% |
| 0.08 | Atomization | 3.51 | 3.42 | 3.49 | 3.31 | 3.37 | 3.25 | 3.18 | 2.45 | 19.8% |
| | Amount | | | | | | | | | |
| | CV | 4.9% | 1.7% | 3.8% | 3.4% | 6.9% | 4.3% | 8.6% | 45.3% | 5.9% |
| 0.12 | Atomization | 3.45 | 3.47 | 3.43 | 3.27 | 3.16 | 2.83 | 2.69 | 2.25 | 24.2% |
| | Amount | | | | | | | | | |
| | CV | 6.3% | 2.2% | 4.6% | 1.9% | 5.7% | 3.1% | 10.3% | 42.7% | 6.7% |
| 0.15 | Atomization | 3.42 | 3.40 | 3.35 | 3.03 | 2.88 | 2.57 | 2.28 | 2.01 | 29.2% |
| | Amount | | | | | | | | | |
| | CV | 7.2% | 1.7% | 3.5% | 5.8% | 2.6% | 4.2% | 12.5% | 36.9% | 7.5% |
| 0.2 | Atomization | 3.48 | 3.19 | 2.88 | 2.67 | 2.42 | 2.19 | 1.93 | 1.72 | 36.8% |
| | Amount | | | | | | | | | |
| | CV | 9.3% | 3.7% | 5.6% | 3.9% | 4.8% | 6.3% | 7.7% | 37.2% | 6.2% |

It can be seen from the test results that within the density range of 0.04 to 0.20 g/cm³, the smaller the density of the liquid storage element 100 is, the smaller the attenuation of the atomization amount during the suction is. Especially when the density is 0.04 to 0.12 g/cm³, the atomization amount of the first 350 puffs is quite stable. When the density is 0.20 g/cm³, there is a significant attenuation even at the first 350 puffs. It is generally believed that the attenuation of the atomization amount during the suction is smaller, the taste is more stable and the user experience is better. The testing data also shows that the attenuation of the atomization amount attenuates is larger at 351-400 puffs, and the CV is significantly larger, which is generally considered to be caused by the unstable voltage generated when the lithium battery is about to be exhausted.

When the density of the liquid storage element 100 is 0.15 g/cm³, the atomization amount at 301-350 puffs is attenuated by 33.3% compared with that at 1-50 puffs, and the atomization amount at 351-400 puffs is attenuated by 41.2% compared with that at 1-50 puffs; when the density of the liquid storage element 100 is 0.20 g/cm³, the atomization amount at 301-350 puffs is attenuated by 44.5% compared with that at 1-50 puffs, and the atomization amount at 351-400 puffs is attenuated by 50.6% compared with that at 1-50 puffs. During the suction, when the atomization amount is reduced by nearly 50% compared with the initial suction stage, it is generally considered that the taste is significantly affected.

Due to the action of the capillary force, a part of the liquid will remain in the liquid storage element 100 after the suction is finished. The lower the residual rate of the liquid. the higher the utilization efficiency of the liquid. It can be seen from the test results that within the density range of 0.04 to 0.20 g/cm³, the smaller the density of the liquid storage element 100 is, the lower the residual rate of the liquid after 400 puffs. When the density of the liquid storage element 100 is 0.04 to 0.12 g/cm³, the residual rate of the liquid after 400 puffs is between 16.5% and 24.2%; when the density of the liquid storage element 100 is 0.15 g/cm³, the residual rate of the liquid after 400 puffs is close to 30%; when the density of the liquid storage element 100 is 0.20 g/cm³, the residual rate of the liquid after 400 puffs exceeds 35%, and the utilization efficiency of the liquid is less than 65%, the waste is more serious.

Comprehensively considering the stability of the atomization amount during the suction and the residual rate of the liquid after the suction and the convenience of assembly, preferably the present invention determines that the density range of the liquid storage element 100 is 0.03 to 0.15 g/cm³, most preferably 0.04 to 0.12 g/cm³.

### <Bicomponent Fibers>

As shown in FIGS. 1a and 1b, the liquid storage element 100 according to the present embodiment has a three-dimensional network structure formed by thermally bonding bicomponent fibers 2, and the bicomponent fibers 2 have a sheath 21 and a core 22. The bicomponent fibers 2 may adopt bonding agent, plasticizer, or heat to bond fibers, preferably adopt heat to bind fibers to avoid the introduction of impurities during manufacturing the liquid storage element 100. The fiber components described in the present invention refer to the polymers that make fibers. Additives for the surface of the fibers, such as surfactants, are not considered to be the fiber components. The liquid storage element 100 of the present embodiment can be soaked through the stored liquid therein, and can change the ability of the liquid storage element 100 to be soaked by the liquid through adding the surfactants.

FIG. 1c shows an enlarged cross-sectional schematic view of bicomponent fibers of FIG. 1a and FIG. 1b. As shown in FIG. 1c, the sheath 21 and the core 22 are concentric structure. The bicomponent fibers 2 with the concentric structure have a greater rigidity, are easy to produce, and are lower in price.

FIG. 1d shows another enlarged cross-sectional schematic view of the bicomponent fibers

of FIG. 1a and FIG. 1b. As shown in FIG. 1d, the sheath 21 and the core 22 are eccentric structure. The bicomponent fibers 2 with an eccentric structure are relatively soft and fluffy, and it is easy to manufacture the liquid storage element 100 having a lower density. In addition, the liquid storage element 100 can be manufactured by using the bicomponent fibers with a coordinate structure, but thermal bonding is difficult. Of course, the liquid storage element 100 can be manufactured by using three-component sheath-and-core structural fibers, but the three-component sheath-and-core structural fibers are difficult to manufacture, have high cost and are poorer in cost performance.

The bicomponent fibers 2 are filaments or staple fibers. The liquid storage element 100 made from filaments has a higher strength, and the liquid storage element 100 made from short fibers has a better elasticity. The manufacturer can select suitable bicomponent fibers to make the liquid storage element 100 with a suitable density and suitable shape according to the performance requirements of the liquid storage element 100.

The core 22 of the bicomponent fibers 2 has a higher melting point than the sheath 21 by 25° C. or more. The liquid storage element 100 of the present embodiment is made by thermally bonding the bicomponent fibers 2 with a sheath-and-core structure, and the core 22 of the bicomponent fibers 2 has a higher melting point than the sheath 21 by 25° C. or more, so that the core 22 can maintain a certain rigidity when thermal bonding is performed between the fibers, which is convenient to manufacture the liquid storage element 100 having a lower density.

The sheath 21 is polyethylene, polypropylene, polyolefin, or copolyester, and the core 22 is a polymer. Alternatively, the sheath 21 is polylactic acid, and the core 22 is polylactic acid having a higher melting point than the sheath 21 by 25° C. or more.

The sheath 21 of the bicomponent fibers 2 can be common polymer or other polyolefins, for example the common polymer is polyethylene, polypropylene, copolyester of polyethylene terephthalate, polyamide-6, polylactic acid, etc. The polyolefins are polymer of olefins, which are a general term for a class of thermoplastic resins usually obtained by independently polymerizing or copolymerizing α-olefins such as ethylene, propylene, 1-butene, 1-pentene, and 1-hexene and the like. The polyolefins have an inert molecular structure, which does not contain active groups on the molecular chain, and hardly reacts with liquid components in the application field of the present invention, so that it has unique advantages.

When the sheath 21 is polyethylene, such as linear low-density polyethylene, low-density polyethylene or high-density polyethylene, the core 22 may be polymer such as polypropylene, polyethylene terephthalate, etc. When the sheath 21 is polypropylene and polyolefin. the core 22 may be polyethylene terephthalate (PET for short), polytrimethylene terephthalate (PTT for short) or polybutylene terephthalate (PBT for short), polyamide, etc. The sheath 21 of the bicomponent fibers 2 has a low melting temperature, which is beneficial to improve the production efficiency and reduce the energy consumption in the manufacturing process.

When the sheath 21 is polylactic acid, according to the melting point of polylactic acid, if the sheath 21 adopts polylactic acid with a melting point of about 130° C., the core 22 may be polypropylene, polyethylene terephthalate, polylactic acid with a melting point of about 170° C. etc. When the sheath 21 is polylactic acid with a melting point of about 170° C., the core 22 may be polyethylene terephthalate, polybutylene terephthalate, polytrimethylene terephthalate, nylon, polyamide, etc. Polylactic acid is a biodegradable material, which can reduce environmental pollution caused by discarding the liquid storage element 100. In particular, when the sheath 21 adopts polylactic acid with a lower melting point, and the core 22 adopts polylactic acid with a higher melting point, the prepared liquid storage element 100 is the biodegradable material.

### <Fineness of Bicomponent Fibers>

The bicomponent fibers 2 from which the liquid storage element 100 of the present invention is made have a fineness of 1-30 denier, preferably 1-15 denier, most preferably 1.5-10 denier. The bicomponent fibers 2 with the sheath-and-core structure which has a fineness less than 1 denier is difficult to manufacture and has high cost. The liquid storage element 100 made from fibers with a fineness higher than 30 denier has insufficient capillary force and is easy to leak. The bicomponent fibers 2 which has the sheath-and-core structure and has 1-15 denier is easily thermally bonded into the liquid storage element 100 which has a lower density and has the three-dimensional structure with a suitable capillary force, and the bicomponent fibers 2 with the sheath-and-core structure having 1.5-10 denier is particularly suitable and has lower cost.

The bicomponent fibers with different fineness can be mixed into the fluid storage element 100 to optimize the fluid storage and release properties or to reduce cost. The cost can also be reduced by incorporating some monocomponent fibers, such as polypropylene fibers, into the bicomponent fibers without affecting the processing and performance of the fluid storage element 100.

In the present embodiment, the bicomponent fibers 2 preferably have a fineness of 1.5 denier, 2 denier, 3 denier or 6 denier, the sheath 21 is polyethylene having a melting point of about 130° C., the core 22 is polypropylene having a melting point of about 165° C. and the liquid storage element 100 has a density of 0.04 to 0.12 g/cm³, so that the liquid storage element 100 has advantages of large liquid storage capacity, uneasy leakage and high release efficiency, etc.

Although the liquid storage element 100 may also be made from monocomponent fibers, such as polypropylene fibers, by bonding with bonding agent, the use of the bonding agent often generally makes it difficult for the liquid storage element 100 to conform related regulations of food or drugs, so that the liquid storage element 100 is not suitable for use in aerosol emission devices such as electronic cigarettes, drug atomization, etc.

As shown in FIGS. 1a, 1b, 1c and 1d, in the present embodiment, the liquid storage element 100 has the three-dimensional network structure formed by thermally bonding the bicomponent fibers 2 with a concentric structure or an eccentric structure. The shape of the liquid storage element 100 is a cylinder, the outer diameter is 9 mm, and the liquid storage element 100 is provided with a liquid storage element through-hole 130 which is an axial through-hole having a diameter of 3.5 mm, and one end of the through-hole is connected to the atomizer and conducts the liquid to the atomizer. The shape and size of the liquid storage element 100 is suitable for use in an emulated cigarette-shaped electronic cigarette, and also suitable for use in miniature electric mosquito-repellent incense and aromatherapy. In the present embodiment, the sheath 21 of the bicomponent fibers 2 can be replaced by polylactic acid having a melting point of about 130° C. and the prepared liquid storage element 100 has similar properties.

### The Second Embodiment

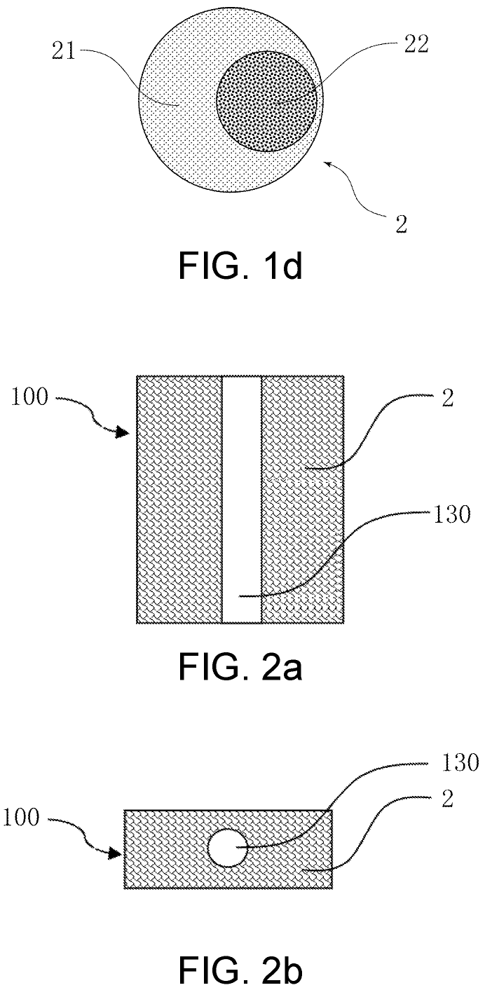

FIG. 2a shows a longitudinal section view of a liquid storage element according to the second embodiment disclosed in the present invention; FIG. 2b shows a cross-sectional view of the liquid storage element according to the second embodiment disclosed in the present invention. The structure of the present embodiment is similar to that of the first embodiment, and the same parts as the first embodiment will not be repeated in the description of this embodiment.

As shown in FIG. 2a and FIG. 2b, in the present embodiment, the liquid storage element 100 has a three-dimensional network structure formed by thermally bonding bicomponent filaments with a concentric structure. The bicomponent fibers 2 have a fineness of 6 denier, and the sheath 21 is polypropylene having a melting point of about 165° C., and the core 22 is polybutylene terephthalate having a melting point of about 230° C., the liquid storage element 100 has a higher temperature resistance, and the prepared liquid storage element 100 has a density of 0.1 to 0.2 g/cm³, which has a greater rigidity and is suitable for high-speed automated assembly. A shape of the cross-sectional view of the liquid storage element 100 is cuboid, and the liquid storage element 100 is provided with a liquid storage element through-hole 130 which is an axial through-hole having a diameter of 3 mm, one end of the through-hole is connected to the atomizer. the aerosol generated during atomization emits through the liquid storage element through-hole 130. This shape of the liquid storage element 100 is suitable for use in cuboid-shape flat cigarettes, and also suitable for use in electric mosquito-repellent incense and electric heating aromatherapy. In the present embodiment, the sheath 21 of the bicomponent fibers 2 can be replaced by polylactic acid having a melting point of about 170° C. and the prepared liquid storage element 100 has similar properties.

### The Third Embodiment

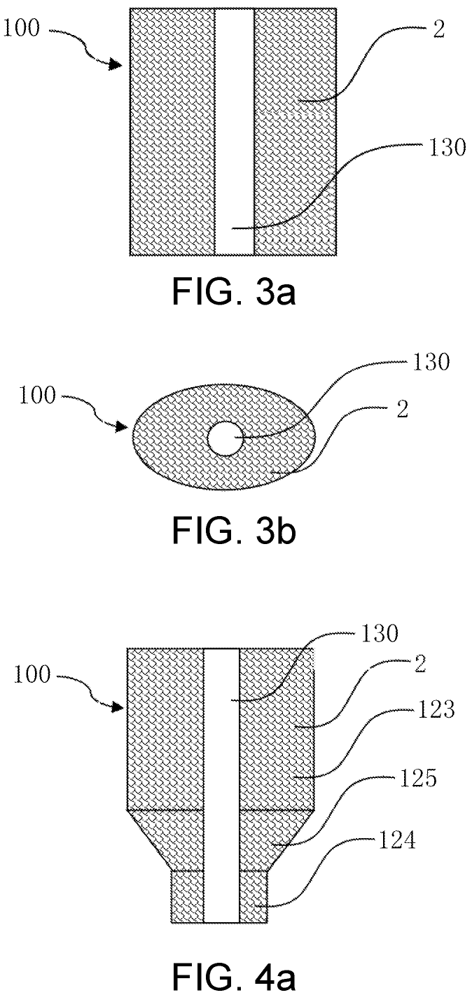

FIG. 3a shows a longitudinal section view of a liquid storage element 100 according to the third embodiment disclosed in the present invention; FIG. 3b shows a cross-sectional view of the liquid storage element 100 according to the third embodiment disclosed in the present invention. The structure of the present embodiment is similar to that of the first embodiment, and the same parts as the first embodiment will not be repeated in the description of this embodiment.

As shown in FIG. 3a and FIG. 3b. in the present embodiment, the liquid storage element 100 has a three-dimensional network structure formed by thermally bonding bicomponent fibers 2 with a concentric structure. The bicomponent fibers 2 are short fibers having a fineness of 2 denier, and the sheath 21 is polylactic acid having a melting point of about 130° C., and the core 22 is polylactic acid having a melting point of 155-185° C. the prepared liquid storage element 100 has a density of 0.08 to 0.12 g/cm³. A shape of the cross-sectional view of the liquid storage element 100 is elliptical, and the liquid storage element 100 is provided with a liquid storage element through-hole 130 which is an axial through-hole having a diameter of 4 mm, one end of the through-hole is connected to the atomizer, the liquid stored in the liquid storage element 100 is conducted to the atomizer through the connection, the aerosol generated during atomization emits through the liquid storage element through-hole 130. This shape of the liquid storage element 100 is suitable for use in elliptical-column-shape flat cigarettes, and also suitable for use in electric mosquito-repellent incense and electric heating aromatherapy which have similar shape. In the present embodiment, the liquid storage element 100 is completely made from polylactic acid, which can be completely biodegraded and is of great significance for reducing environmental pollution.

### The Fourth Embodiment

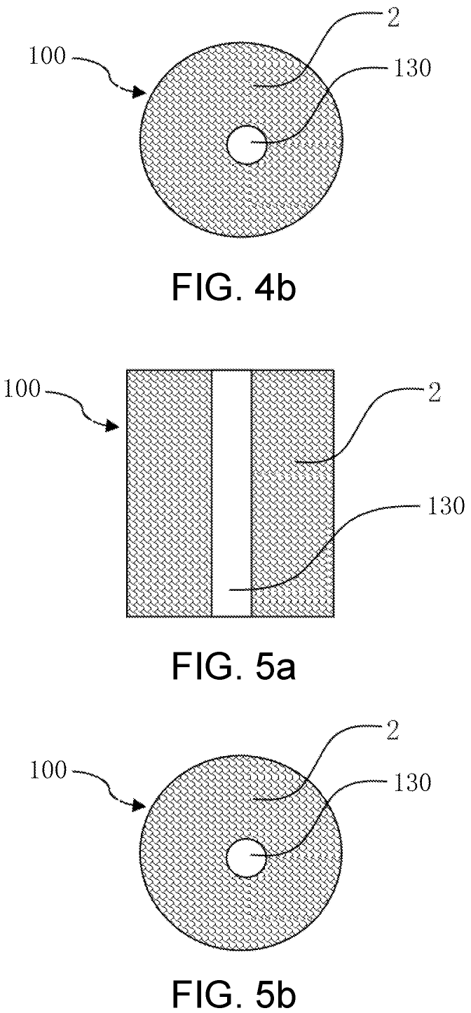

FIG. 4a shows a longitudinal section view of a liquid storage element 100 according to the fourth embodiment disclosed in the present invention; FIG. 4b shows a cross-sectional view of the liquid storage element 100 according to the fourth embodiment disclosed in the present invention. The structure of the present embodiment is similar to that of the first embodiment, and the same parts as the first embodiment will not be repeated in the description of this embodiment.

As shown in FIG. 4a and FIG. 4b, in the present embodiment, the liquid storage element 100 has a three-dimensional network structure formed by thermally bonding bicomponent fibers 2 with an eccentric structure. The bicomponent fibers 2 are short fibers having a fineness of 3 denier, and the sheath 21 is polylactic acid having a melting point of about 130° C., and the core 22 is polyethylene terephthalate. The prepared liquid storage element 100 has a density of 0.03 to 0.06 g/cm³, and has characteristics in that the liquid absorption capacity is large and the release residue is low. The liquid storage element 100 is cylindrical, and is provided with a liquid storage element through-hole 130 which is an axial through-hole having a diameter of 4.5 mm, one end of the through-hole is connected to the atomizer, the aerosol generated during atomization emits through the liquid storage element through-hole 130.

In the present embodiment, a portion of the liquid storage element 100 which connects the atomizer is compressed to have a higher density, and the liquid is enriched to the higher density portion in the process of consumption, thereby improving the uniformity of liquid release and further reducing the liquid residue after application.

Preferably, the liquid storage element 100 is compressed to form a low-density portion 123, a high-density portion 124 and a density increasing portion 125 disposed between the low-density portion 123 and the high-density portion 124. Therefore, the liquid can be better enriched to the high-density portion 124, which can improve the fluency of liquid conduction, and reduce the liquid residue of the liquid storage element 100 after application.

The shape of the liquid storage element 100 is suitable for use in cylindrical electronic cigarettes, and is also suitable for use in electric mosquito-repellent incense and electric aromatherapy. In the present embodiment, the sheath 21 of the bicomponent fibers 2 can be replaced by polyolefin or copolyester of polyethylene terephthalate having a melting point of about 110° C., and the prepared liquid storage element 100 has similar properties.

### The Fifth Embodiment

FIG. 5a shows a longitudinal section view of a liquid storage element 100 according to the fifth embodiment disclosed in the present invention; FIG. 5b shows a cross-sectional view of the liquid storage element 100 according to the fifth embodiment disclosed in the present invention. The structure of the present embodiment is similar to that of the first embodiment, and the same parts as the first embodiment will not be repeated in the description of this embodiment.

As shown in FIG. 5a and FIG. 5b, in the present embodiment. the liquid storage element 100 has a three-dimensional network structure formed by thermally bonding bicomponent fibers 2 with a concentric structure. The bicomponent fibers 2 are filaments having a fineness of 30 denier, and the sheath 21 is copolyester of polyethylene terephthalate having a melting point of about 200° C. and the core 22 is polybutylene terephthalate having a melting point of about 270° C. this liquid storage element 100 has a higher temperature resistance, and the prepared liquid storage element 100 has a density of 0.15 to 0.25 g/cm³, the liquid storage element 100 is cylindrical and is provided with a liquid storage element through-hole 130 which is an axial through-hole having a diameter of 5 mm, one end of the through-hole is connected to the electric heating atomizer or the ultrasonic atomizer, the aerosol generated during atomization emits through the liquid storage element through-hole 130. The liquid storage element 100 is suitable for use in portable electric mosquito-repellent incense or aromatherapy, and also suitable for use in electronic cigarettes. In the present embodiment, the sheath 21 of the bicomponent fibers 2 can be replaced by polylactic acid having a melting point of about 170° C. and the prepared liquid storage element 100 has similar properties.

### The Sixth Embodiment

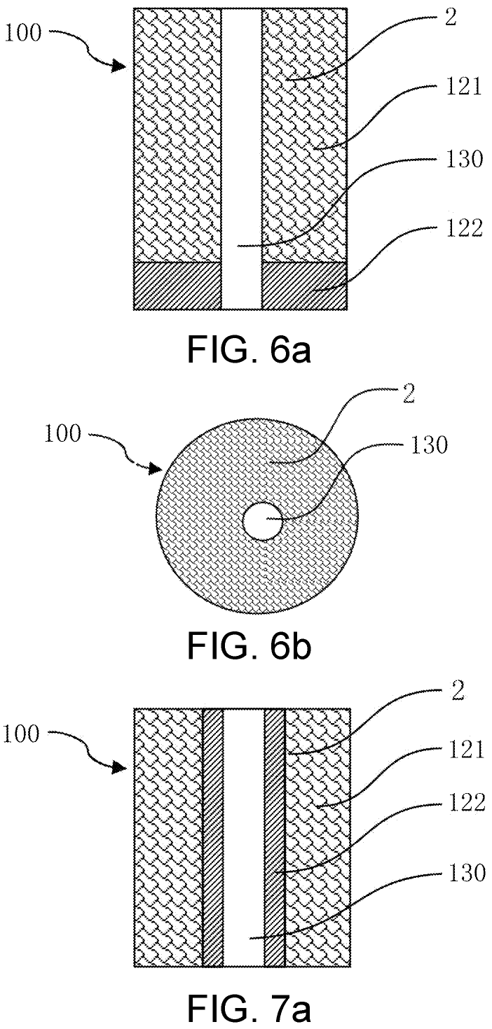

FIG. 6a shows a longitudinal section view of a liquid storage element 100 according to the sixth embodiment disclosed in the present invention; FIG. 6b shows a cross-sectional view of the liquid storage element 100 according to the sixth embodiment disclosed in the present invention. The structure of the present embodiment is similar to that of the first embodiment, and the same parts as the first embodiment will not be repeated in the description of this embodiment.

As shown in FIG. 6a and FIG. 6b, in the present embodiment, the liquid storage element 100 includes a liquid storage portion 121 and a liquid collection portion 122 of an upper and lower structure. Both of the liquid storage portion 121 and the liquid collection portion 122 have a liquid storage element through-hole 130 axially penetrating therethrough.

The liquid storage portion 121 has a three-dimensional network structure formed by thermally bonding bicomponent fibers 2 with an eccentric structure. The bicomponent fibers 2 have a fineness of 3 denier, and the sheath 21 is polyethylene having a melting point of about 130° C., and the core 22 is polypropylene having a melting point of about 165° C. the liquid storage portion 121 has a density of 0.04 to 0.08 g/cm³, and the liquid storage portion 121 is cylindrical. The bicomponent fibers 2 made into the liquid collection portion 122 is same as the fibers made into the liquid storage portion 121, both the liquid storage portion 121 and the liquid collection portion 122 are provided with the liquid storage element through-hole 130 which is an axial through-hole having a diameter of 4 mm, one end of the through-hole is connected to the electric heating atomizer or the ultrasonic atomizer, the aerosol generated during atomization emits through the liquid storage element through-hole 130. The liquid storage element 100 is suitable for use in portable electric mosquito-repellent incense or aromatherapy, and also suitable for use in electronic cigarettes. In the present embodiment, the sheath 21 of the bicomponent fibers 2 can be replaced by polylactic acid having a melting point of about 130° C., and the prepared liquid storage element 100 has similar properties.

In the present embodiment, the density of the liquid collection portion 122 is higher than that of the liquid storage portion 121. Because the liquid collection portion 122 has a higher density than the liquid storage portion 121. the liquid is enriched to the liquid collection portion 122 with a higher density during the process of consumption, thereby improving the fluency of liquid release, and further reducing the liquid residue after application.

### The Seventh Embodiment

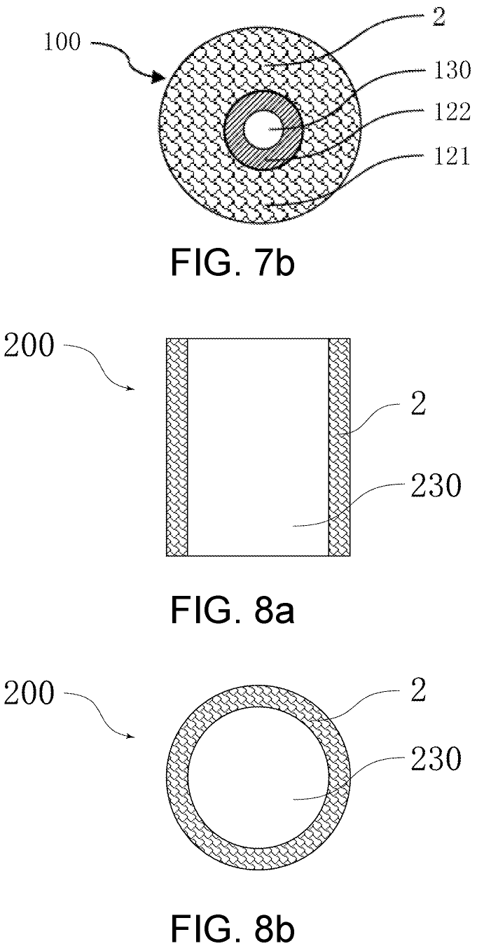

FIG. 7a shows a longitudinal section view of a liquid storage element 100 according to the seventh embodiment disclosed in the present invention; FIG. 7b shows a cross-sectional view of the liquid storage element 100 according to the seventh embodiment disclosed in the present invention. The structure of the present embodiment is similar to that of the first embodiment, and the same parts as the first embodiment will not be repeated in the description of this embodiment.

As shown in FIG. 7a and FIG. 7b, in the present embodiment, the liquid storage element 100 includes a liquid storage portion 121 and a liquid collection portion 122, the liquid storage portion 121 coats on peripheral wall of the liquid collection portion 122 and has a lower density than the liquid collection portion 122. The liquid collection portion 122 have a liquid storage element through-hole 130 axially penetrating therethrough.

The liquid storage portion 121 has a three-dimensional network structure formed by thermally bonding bicomponent fibers 2 with a concentric structure. The bicomponent fibers 2 have a fineness of 3 denier, and the sheath 21 is polylactic acid having a melting point of about 130° C., and the core 22 is polyethylene terephthalate having a melting point of about 270° C., the liquid storage portion 121 has a density of 0.1 to 0.15 g/cm³, the liquid storage portion 121 is cylindrical. The liquid collection portion 122 has a three-dimensional network structure formed by thermally bonding bicomponent fibers 2 with a concentric structure. The bicomponent fibers 2 have a fineness of 2 denier, and the sheath 21 is polylactic acid having a melting point of about 170° C., and the core 22 is polyethylene terephthalate having a melting point of about 270° C. The liquid collection portion 122 have an aerosol channel which is an axial through-hole having a diameter of 4 mm, one end of the through-hole is connected to the electric heating atomizer or the ultrasonic atomizer, the aerosol generated during atomization emits through the liquid storage element through-hole 130. The liquid storage element 100 is suitable for use in portable electric mosquito-repellent incense and aromatherapy, and also suitable for use in electronic cigarettes.

In the present embodiment, the density of the liquid collection portion 122 is higher than that of the liquid storage portion 121. Because the liquid collection portion 122 has a higher density than the liquid storage portion 121, the liquid is enriched to the liquid collection portion 122 with a higher density during the process of consumption, thereby improving the fluency of liquid release, and further reducing the liquid residue after application.

In summary, the liquid storage element 100 according to the present invention which used in the aerosol emission device is made from bicomponent fibers with a sheath-and-core structure, and is provided with an aerosol channel in the axial direction, the aerosol channel is formed by the liquid storage element through-hole 130 of the liquid storage element 100. The liquid storage element 100 of the present invention can be widely applied to various aerosol emission devices that vaporize or atomize the liquid to store and release the liquid, thereby improving user experience. The liquid storage element 100 can be made into the required size and shape of the three-dimensional structure during the thermal bonding process according to the application requirements, so that the liquid storage element 100 is suitable for high-speed automated assembly, reducing the manufacturing cost of aerosol emission devices, such as electronic cigarettes, drug atomization, electric mosquito-repellent incense and electric aromatherapy, etc. The foregoing embodiments of the present invention are only intended to illustrate the principle and advantages of the present invention rather than limiting the present invention. Those skilled in the art can make modifications or changes to the foregoing embodiments without departing from the spirit and scope of the present invention. Therefore, all equivalent modifications or changes made by those skilled in the art without departing from the spirit and technical concepts disclosed by the present invention shall still be covered by the claims of the present invention.

### The Eighth Embodiment

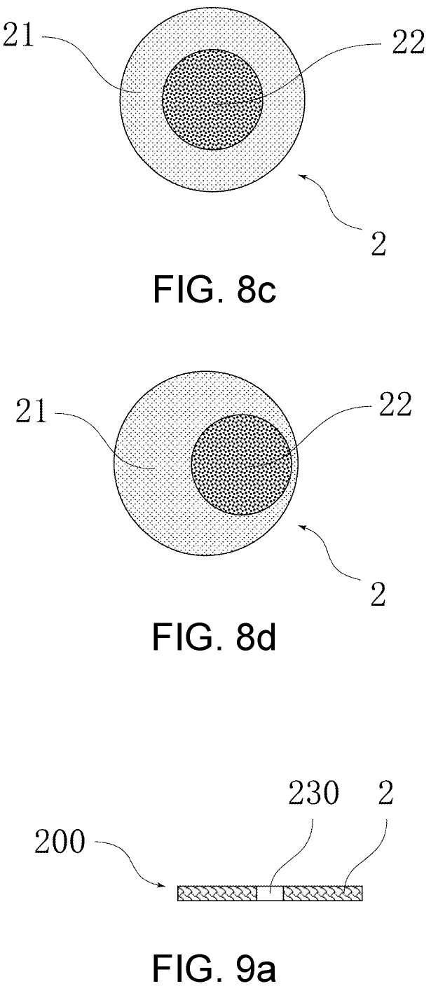

FIG. 8a shows a longitudinal section view of a wicking element according to the eighth embodiment disclosed in the present invention; FIG. 8b shows a cross-sectional view of the wicking element according to the eighth embodiment disclosed in the present invention; FIG. 8c shows an enlarged cross-sectional schematic view of bicomponent fibers of FIGS. 8a and 8b; FIG. 8d shows another enlarged cross-sectional schematic view of the bicomponent fibers of FIGS. 8a and 8b.

As shown in FIGS. 8a to 8b, a wicking element 200 according to the eighth embodiment of the present invention is used for conducting liquids in an aerosol emission device. The wicking element 200 has a three-dimensional network structure formed by thermally bonding bicomponent fibers 2, the bicomponent fibers 2 have a skin 21 and a core 22.

### <Shape, Thickness, Rigidity and Speed of Liquid Penetration of Wicking Element>

The wicking element 200 may have a wicking element through-hole 230 axially penetrating therethrough.

According to the design of the aerosol emission device, the wicking element 200 of the present embodiment can be designed as a sheet shape or a tube shape. As shown in FIGS. 8a and 8b, the wicking element 200 of the present embodiment is designed as a tube shape.

The wicking element 200 can also be designed as a sheet shape. The sheet-shaped wicking element 200 may also be provided with the wicking element through-hole 230.

According to the structure of the aerosol emission device, the cross section of the wicking element 200 can be made into a circular ring, an elliptical ring or other desired shapes.

For the sheet-shaped wicking element 200, an axial direction herein is defined as a thickness direction thereof, and a radial direction is defined as a direction perpendicular to the thickness. By adopting appropriate fabrication techniques, the fibers in the wicking element 200 can have more axial alignment orientations, in this case, an axial rigidity of the sheet-shaped wicking element 200 is greater than its radial rigidity, the speed of liquid penetration along the axial direction in the wicking element 200 is greater than the speed of liquid penetration along the radial direction; it is also possible to that the fibers in the wicking element 200 can have more radially alignment orientations, in this case, a radial rigidity of the sheet-shaped wicking element 200 is greater than its axial rigidity, and the speed of liquid permeation along the radial direction in the wicking element 200 is greater than the speed of liquid permeation along the axial direction.

For the tube-shaped wicking element 200, an axial direction herein is defined as a direction of a central axis of the wicking element through-hole 230, and a radial direction is defined as a direction perpendicular to the central axis of the wicking element through-hole 230. The fibers in the tube-shaped wicking element 200 have more axial alignment orientations, an axial rigidity of the wicking element 200 is greater than its radial rigidity, and the speed of liquid penetration along the axial direction in the wicking element 200 is greater than the speed of liquid penetration along its radial direction.

The rigidity comparison method herein is: placing the wicking element 200 in the axial or radial direction, clamping it between two parallel plates, then measuring an axial height or a radial height of the wicking element 200 before compression; under the condition of applying the same acting force, measuring an axial height or a radial height after the two plates axially or radially compress the wicking element 200, and calculating a deformation amount of the compression which is the difference between the axial height or the radial height before compression and the axial height or the radial height after compression; and dividing the deformation amount of the compression by the axial height or the radial height of the wicking element 200 before compression to obtain a compression ratio. The smaller the compression ratio, the higher the rigidity, and the greater the compression ratio, the less the rigidity.

The thickness of the wicking element 200 refers to the shortest distance that the liquid conducts from one side of the wicking element 200 to the other side, the thickness of the tube-shaped wicking element 200 refers to the thickness of the tube wall, and the thickness of the sheet-shaped wicking element 200 refers to the thickness in the thickness direction thereof.

The thickness of the wicking element 200 is 0.3 mm-3 mm, preferably 0.6 mm, 0.9 mm, 1.2 mm, 1.5 mm, 2 mm. When the thickness of the wicking element 200 is less than 0.3 mm, it is difficult to manufacture a uniform wicking element 200, and it is also inconvenient to install. When the thickness of the wicking element 200 is greater than 3 mm, the wicking element 200 occupies too much space in the aerosol emission device, especially for the tube-shaped wicking element 200, when the thickness of the wicking element 200 is greater than 3 mm, it is generally difficult to install in a fine aerosol emission device. In addition, when the thickness of the wicking element 200 is greater than 3 mm, the wicking element 200 absorbs too much liquid, which affects the utilization efficiency of the liquid.

### <Density of Wicking Element>

The density of the wicking element 200 of the present embodiment is 0.05 to 0.35 g/cm³. preferably 0.1 to 0.3 g/cm³. When the density is less than 0.05 g/cm³, the strength of the wicking element 200 is insufficient, and the tube-shaped wicking element 200 is easily deformed or even wrinkled when assembled with the aerosol emission device, which affects the stability of atomization, and even causes liquid leakage in severe cases. When the density is greater than 0.35 g/cm³, the liquid conducting speed is slow, the atomization efficiency is affected, and the hardness of the high-density wicking element is too high, the radial elasticity is insufficient, the matching performance between the tube-shaped wicking element and the aerosol emission device is reduced.

### <Bicomponent Fibers>

FIG. 8c shows an enlarged cross-sectional schematic view of bicomponent fibers of FIGS. 8a and 8b. As shown in FIG. 8c. the sheath 21 and the core 22 are concentric structure. FIG. 8d shows another enlarged cross-sectional schematic view of the bicomponent fibers of FIGS. 8a and 8b. As shown in FIG. 8d. the sheath 21 and the core 22 are eccentric structure. The wicking element 200 made of the bicomponent fibers 2 with the concentric structure have a greater rigidity, the wicking element 200 made of the bicomponent fibers 2 with the eccentric structure has better elasticity.

The bicomponent fibers 2 are filaments or staple fibers. The wicking element 200 made from filaments has a greater rigidity, and the wicking element 200 made from short fibers has a better elasticity. The suitable wicking element 200 can be made by selecting suitable bicomponent fibers according to the performance requirements of the wicking element 200.

The core 22 of the bicomponent fibers 2 has a higher melting point than the sheath 21 by 20° C. or more. The wicking element 200 of the present embodiment is made by thermally bonding the bicomponent fibers 2 with a sheath-and-core structure, and the core 22 of the bicomponent fibers 2 has a higher melting point than the sheath 21 by 20° C. or more, so that the core 22 can maintain a certain rigidity when thermal bonding is performed between the fibers, which is convenient to manufacture the wicking element 200 with uniform voids.

The sheath 21 of the bicomponent fibers 2 can be polyolefin. polyethylene terephthalate copolyester, polypropylene terephthalate, polybutylene terephthalate, polylactic acid or polyamide-6. The polyolefins are polymer of olefins, which are a general term for a class of thermoplastic resins usually obtained by independently polymerizing or copolymerizing α-olefins such as ethylene, propylene, 1-butene, 1-pentene, and 1-hexene and the like. It can also be a common polymer such as polyester or low melting point copolyester.

When the sheath 21 is polyethylene, the core 22 can be polypropylene, polyethylene terephthalate (referred to as PET) and other polymers. When the sheath 21 is polypropylene, the core 22 can be PET, polyamide, etc. The sheath 21 of the bicomponent fibers 2 has a low melting temperature, which is beneficial to improve the production efficiency and reduce manufacturing costs. The sheath 21 of the bicomponent fibers 2 has a high melting temperature, making the wicking element 200 have high temperature resistance, which is conducive to increasing the working temperature of the atomizer.

When the sheath 21 is polylactic acid, the core 22 can be polypropylene. polyethylene terephthalate, poly L-lactic acid or poly D-lactic acid with melting point 155-180° C., etc. if the sheath 21 adopts poly D, L-lactic with a melting point of 125-135° C. according to the melting point of polylactic acid. When the sheath 21 is poly L-lactic acid or poly D-lactic acid with melting point 145-180° C., the core 22 can be polyethylene terephthalate. polybutylene terephthalate (referred to as PBT), polypropylene terephthalate (referred to as PTT), polyamide, etc. Polylactic acid is a biodegradable material, which can reduce environmental pollution caused by discarding the wicking element 200.

When the sheath 21 is polyester or copolyester, the core 22 can be selected according to the melting point of the sheath 21. For example, the sheath 21 can use PBT or PTT with a melting point of 225-235° C., and the core 22 can use PET with a melting point of 255-265° C. Another example is that the sheath 21 is copolyester of polyethylene terephthalate (referred to as Co-PET) with a melting point of 110-120° C. or 160-200° C., and the core can be PET, PBT or PTT.

The bicomponent fibers 2 from which the wicking element 200 of the present invention is made have a fineness of 1-30 denier, preferably 1.5-10 denier. The bicomponent fibers 2 with the sheath-and-core structure which has a fineness less than 1 denier is difficult to manufacture and has high cost. The wicking element 200 made from fibers with a fineness higher than 30 denier has insufficient capillary force, and the liquor conduction is poor. The bicomponent fibers 2 which has the sheath-and-core structure and has 1-15 denier is easily to make the wicking element 200, and the bicomponent fibers 2 with the sheath-and-core structure having 1.5-10 denier is particularly suitable and has lower cost. When the viscosity of the atomized liquid is low, it is advisable to use fibers with smaller denier to make wicking element 200, such as fibers of 1 denier. 1.5 denier, 2 denier, and 3 denier. When the viscosity of the atomized liquid is high, it is advisable to use fibers with larger denier to make wicking element 200, such as 6 denier, 10 denier, 30 denier fibers.

As shown in FIGS. 8a, 8b, 8c and 8d, in the present embodiment, the wicking element 200 is preferably made from staple bicomponent fibers 2 with concentric structure to form a tubular structure, which has a three-dimensional network structure formed by thermally bonding bicomponent fibers. The sheath 21 is polyethylene with a melting point of 125-135° C., the core 22 is polypropylene with a melting point of 160-170° C., and the density of the wicking element 200 is between 0.05 to 0.35 g/cm³. The wicking element 200 has good axial strength, good radial elasticity, and faster liquid conduction speed. The wicking element 200 can be used for the atomization of electronic cigarette liquid, which is also suitable for use in mini electric mosquito coils and aromatherapy.

In the present embodiment, the core of the bicomponent fibers 2 can be made of PET, PBT, PTT, polyamide, etc. when the sheath 21 of the bicomponent fibers 2 is replaced by polypropylene with a melting point of 160-170° C. The wicking element 200 has high temperature resistance. It is also possible to use PBT or PTT as the sheath, and PET as the core to make a higher temperature resistant wicking element 200.

In another preferred method in the present embodiment, the wicking element 200 is thermally bonded by bicomponent fiber with an eccentric structure to form a three-dimensional network of tubular structures. The sheath 21 of the wicking element 200 is polyethylene, the core 2 is polypropylene or PET. The thickness of the wicking element 200 is 0.3 to 0.8 mm, and the density of the wicking element 200 is 0.1 to 0.3 g/cm³.

### The Ninth Embodiment

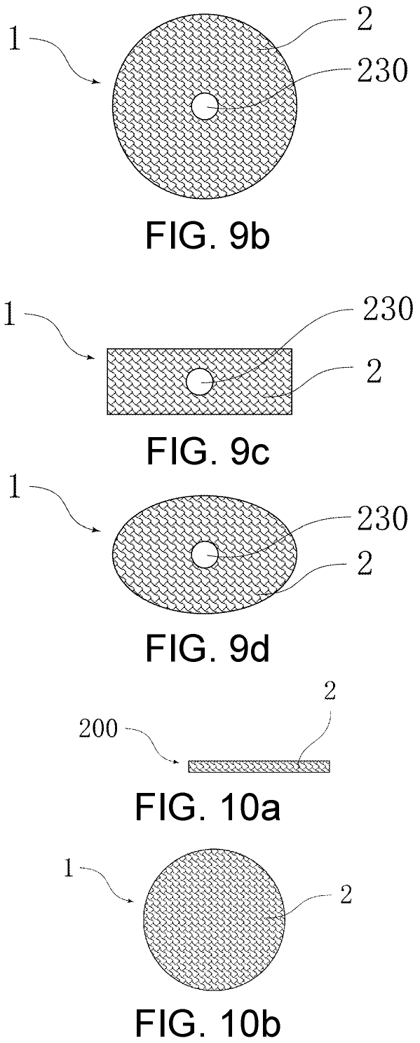

FIG. 9a shows a longitudinal section view of a wicking element according to the ninth embodiment disclosed in the present invention. FIG. 9b shows a cross-sectional view of the wicking element according to the ninth embodiment disclosed in the present invention when the wicking element is cylinder. FIG. 9c shows a cross-sectional view of the wicking element according to the ninth embodiment disclosed in the present invention when the wicking element is cuboid. FIG. 9d shows a cross-sectional view of the wicking element according to the ninth embodiment disclosed in the present invention when the wicking element is elliptic cylinder. The structure of the present embodiment is similar to that of the eighth embodiment, and the same parts as the eighth embodiment will not be repeated in the description of this embodiment.

In the present embodiment, the wicking element 200 is sheet shape, and has a three-dimensional network sheet-shaped structure formed by thermally bonding bicomponent fibers 2 with a concentric structure. The thickness of the wicking element 200 is 0.8-1.5 mm, and the center thereof is provided with a wicking element through-hole 230. The sheath 21 of the wicking element 200 is poly-D,L-lactic acid having a melting point of 125-135° C., and the core 22 is poly-L-lactic acid or poly-D-lactic acid having a melting point of 155-180° C. the prepared wicking element 200 has a density of 0.2 to 0.3 g/cm³, the wicking element 200 is a biodegradable material, which can reduce the environmental pollution caused by discarding the wicking element 200.

In the present embodiment. the radial rigidity of the sheet-shaped wicking element 200 is greater than its axial rigidity, and the speed of liquid permeating along the radial direction in the wicking element 200 is greater than the speed of liquid permeating along the axial direction.

As shown in FIGS. 9b, 9c and 9d, according to the structure of the aerosol emission device, the wicking element 200 can be designed as a cylinder, a square cylinder and an elliptical cylinder, respectively, and the corresponding cross sections are a circular ring, a square ring, and an elliptical ring, respectively. It can also be designed into other desired shapes according to requirements.

### The Tenth Embodiment

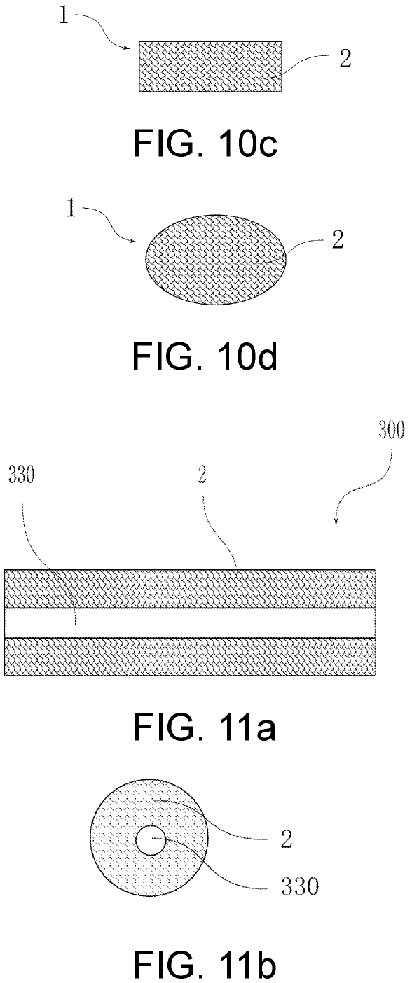

FIG. 10a shows a longitudinal section view of a wicking element according to the tenth embodiment disclosed in the present invention. FIG. 10b shows a cross-sectional view of the wicking element according to the tenth embodiment disclosed in the present invention when the wicking element is cylinder. FIG. 10c shows a cross-sectional view of the wicking element according to the tenth embodiment disclosed in the present invention when the wicking element is cuboid. FIG. 10d shows a cross-sectional view of the wicking element according to the tenth embodiment disclosed in the present invention when the wicking element is elliptic cylinder. The structure of the present embodiment is similar to that of the eighth embodiment, and the same parts as the eighth embodiment will not be repeated in the description of this embodiment.

In the present embodiment, the wicking element 200 is sheet shape, the center thereof doesn't provide with a wicking element through-hole 230, and the wicking element 200 has a three-dimensional network structure formed by thermally bonding bicomponent fibers 2 with an eccentric structure. The sheath 21 is poly-D-lactic acid or poly-L-lactic acid having a melting point of 145-180° C., and the core 22 is PET having a melting point of 255-265° C. the prepared wicking element 200 has a density of 0.25 to 0.35 g/cm³ and has a thickness of 3 mm. The wicking element 200 has a relatively high liquid conducting velocity. In the present embodiment, the sheath of the bicomponent fibers can be replaced by Co-PET to reduce the cost, or replaced by PBT or PTT, so that the wicking element 200 has a better temperature resistance performance.

In the present embodiment, the axial rigidity of the wicking element 200 is greater than its radial rigidity, and the speed of liquid permeating along the axial direction in the wicking element 200 is greater than the speed of liquid permeating along the radial direction.

In the present embodiment, also, the wicking element 200 may be a three-dimensional network sheet-shaped structure formed by thermally bonding bicomponent fibers 2 with a concentric structure, and has a thickness of 1.5-2 mm. The sheath 21 of the wicking element is PBT or PTT, the core 22 is PET, and the prepared wicking element 200 has a density of 0.25 to 0.35 g/cm³. Preferably, the radial rigidity of the sheet-shaped wicking element is greater than its axial rigidity, and the speed of liquid permeation along the radial direction in the wicking element is greater than the speed of liquid permeation along the axial direction.

As shown in FIGS. 10b, 10c and 10d, according to the structure of the aerosol emission device, the wicking element 200 can be designed as a cylinder, a square cylinder and an elliptical cylinder, respectively, and the corresponding cross sections are a circle, a rectangle and an ellipse, respectively. It can also be designed into other desired shapes according to requirements.

In summary, the wicking element for aerosol emission device according to the present invention is made by bonding bicomponent fibers, which can be widely used in various aerosol emission devices. The wicking element has good strength, suitable for automatic assembly, and greatly improves the production efficiency of the aerosol emission device. The wicking element can smoothly and quickly conduct the liquid to the atomizer, improving the atomization efficiency and stability. The foregoing embodiments of the present invention are only intended to illustrate the principle and advantages of the present invention rather than limiting the present invention. Those skilled in the art can make modifications or changes to the foregoing embodiments without departing from the spirit and scope of the present invention. Therefore, all equivalent modifications or changes made by those skilled in the art without departing from the spirit and technical concepts disclosed by the present invention shall still be covered by the claims of the present invention.

### The Eleventh Embodiment

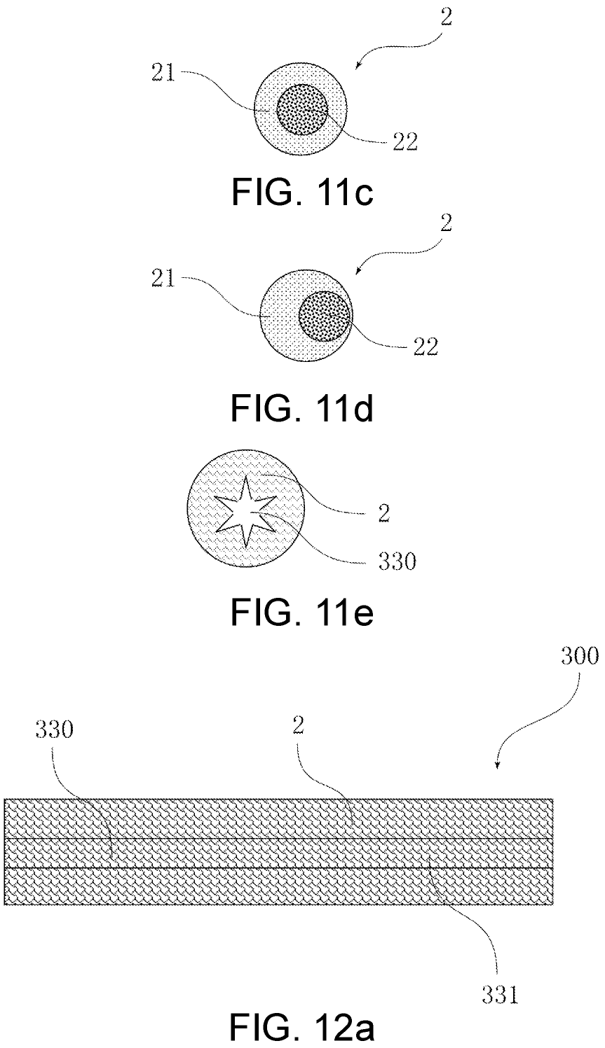

FIG. 11a shows a longitudinal section view of a cooling element according to the eleventh embodiment of the present invention. FIG. 11b shows a cross-sectional view of the cooling element according to the eleventh embodiment of the present invention. FIG. 11c shows an enlarged cross-sectional schematic view of bicomponent fibers of FIGS. 11a and 11b. FIG. 11d shows another enlarged cross-sectional schematic view of the bicomponent fibers of FIGS. 11a and 11b. FIG. 11e shows another cross-sectional view of the cooling element according to the eleventh embodiment of the present invention.

As shown in FIGS. 11a to 11e, a cooling element according to the eleventh embodiment of the present invention is used for cooling an aerosol generated in an aerosol emission device, and the cooling element 300 has a three-dimensional network structure formed by thermally bonding bicomponent fibers 2, the bicomponent fibers 2 have a sheath 21 and a core 22. The present embodiment is applicable to various aerosol emission devices, such as heat-not-burn-type aerosol emission devices and atomizing-type aerosol emission devices. The aerosol emission device includes an aerosol substrate, and the aerosol substrate comprises an aerosol agent, such as propylene glycol, glycerin, water, and the like. The aerosol substrate may also comprise a carrier, such as tobacco, Chinese herbal medicine, fibers, paper scraps, and the like. When a traditional cigarette is burned, the temperature is as high as about 800° C. so that most of the moisture in the tobacco is evaporated, the aerosol is relatively dry, the temperature sensed by the user when inhales the aerosol is lower. Differently, when the aerosol emission device is heating, the temperature is lower, only 200-400° C., and the generated aerosol may contain higher moisture and contain a vaporized aerosol agent, such as propylene glycol, glycerin, and the like, the temperature sensed by the user when inhales the aerosol is higher. Therefore, cooling the aerosol to a temperature that the user feels comfortable and removing the condensate is an important consideration for an aerosol emission device.

In the atomization-type aerosol emission device, the aerosol substrate can also be a liquid storage element loaded with an aerosol agent. In this case, the aerosol agent in the aerosol substrate 891 is heated by the heating element (not shown) and then atomized and cooled by the cooling element 300 and then escapes. Meanwhile, the cooling element 300 also has the function of absorbing the condensate in the aerosol, so that the temperature sensed by the user when inhales the aerosol is moderate, and the aerosol basically does not contain the condensate, which improves the taste and experience.

### <Porosity of Cooling Element>

The cooling element 300 according to the present embodiment formed by thermally

bonding bicomponent fibers can be made with different porosity. In the present embodiment, the porosity of the cooling element 300 may be set to 65%-95%, and preferably 75-85%.

When the porosity is greater than 95%, the cooling element 300 is difficult to form and has insufficient hardness. When the porosity is less than 65%, the hardness of the cooling element 300 is too large, or the cost is too high, it is not suitable for use in the aerosol emission device.

### <Structure of Cooling Element>

The cooling element 300 according to the present embodiment can be made into various structures as required. As shown in FIGS. 11a to 11c, the cooling element 300 may be configured as a hollow structure, that is, the cooling element 300 may have a cooling element through-hole 330 axially penetrating therethrough.

As shown in FIG. 11b, the cooling element 300 may be configured as a hollow structure in which the cross section of the cooling element through-hole 330 is a circular, the cross section of the cooling element 300 is a circular ring. Alternatively, as shown in FIG. 11e, the cooling element 300 may be configured as a hollow structure which the cross section of the cooling element through-hole 330 is a star, the cross sectional of the cooling element 300 is a star ring, and the cross section of the cooling element through-hole 330 may be a pentagram, a hexagram, etc.

When manufacturing the cooling element 300, the hollow cooling element 300 and the non-hollow cooling element 300 can be used alone or in combination to achieve appropriate cooling effect and control a suitable air resistance.

In the present embodiment, employing the cooling element 300 with a hollow structure can reduce the resistance of the aerosol passing through the cooling element 300, so that the high-temperature aerosol passes through a hollow channel with a low air resistance, and when the inner surface of the hollow channel is in contact with the high-temperature aerosol, the sheath 21 of the bicomponent fibers 2 absorbs a large amount of heat from the high-temperature aerosol and then melts, so that the temperature of the aerosol is dropped rapidly. When the high-temperature aerosol mainly passes through the hollow channel, the outer periphery of the cooling element 300 is far away from the high-temperature aerosol, the temperature has dropped to a lower temperature when the temperature is transferred to the outer periphery, so that it can prevent the outer peripheral wall of the cooling element 300 from being deformed due to high temperature or prevent the structure and performance of the aerosol emission device from being damaged.

<Bicomponent Fibers>

As shown in FIGS. 11c and 11d, the cooling element 300 according to the present invention has a three-dimensional network structure formed by thermally bonding bicomponent fibers 2, the bicomponent fibers 2 have a sheath 21 and a core 22.

FIG. 11c shows an enlarged cross-sectional schematic view of bicomponent fibers of FIGS. 11a and 11b. As shown in FIG. 11c, the sheath 21 and core 22 are concentric structures. FIG. 11d shows another enlarged cross-sectional schematic view of the bicomponent fibers of FIGS. 11a and 11b. As shown in FIG. 11d, the sheath 21 and core 22 are eccentric structures. When the density is the same, the cooling element 300 made of a bicomponent fibers 2 with concentric structure is more rigid, and the cooling element 300 made of a bicomponent fibers 2 with eccentric structure has better elasticity.

The bicomponent fibers 2 is filament or staple fiber. The cooling element 300 made of filament has greater axial rigidity, and the cooling element 300 made of staple fiber has better radial elasticity. Bicomponent fibers can be selected to make a suitable cooling element 300 according to the performance requirements of the cooling element 300.

The sheath 21 of the bicomponent fibers 2 can be polyethylene, polypropylene and other polyolefins, or copolyester of ethylene terephthalate, polypropylene terephthalate. polybutylene terephthalate, poly D-lactic acid, poly L-lactic acid. poly D, L-lactic acid, or polyamide-6, etc. The polyolefins are polymer of olefins, which are a general term for a class of thermoplastic resins usually obtained by independently polymerizing or copolymerizing a-olefins such as ethylene. propylene, 1-butene, 1-pentene, and 1-hexene and the like. Polyolefins have an inert molecular structure, do not contain active groups on the molecular chain, and virtually do not react with liquid components in the field of application of the present invention, so it has a unique advantage.

The core 22 can be polypropylene, polyethylene terephthalate and other polymers when the sheath layer 21 is polyethylene. The core 22 can be polyethylene terephthalate, polyamide, etc. when the sheath 21 is polypropylene. The sheath 21 of the bicomponent fibers 2 has a low melting temperature, which is beneficial to improve the production efficiency and reduce manufacturing costs. The sheath 21 of the bicomponent fibers 2 has a high melting temperature and the core 22 with a higher melting point is used, which can make a high-temperature cooling section that can withstand higher temperature aerosols.

When the sheath 21 is polylactic acid, the core 22 can be polypropylene. polyethylene

terephthalate, polylactic acid with a melting point of about 170° C., etc. if the polylactic acid with a melting point of about 130° C. is used as the sheath layer 21 according to the melting point of polylactic acid. The core 22 can be polyethylene terephthalate, polybutylene terephthalate, polypropylene terephthalate, polyamide, etc. when the sheath 21 is polylactic acid with a melting point of 150-185° C. Polylactic acid is a biodegradable material, which can reduce the environmental pollution caused by discarding of the cooling element 300. The bicomponent fibers 2 of the cooling element 300 of the present invention have a fineness between 1-30 denier, preferably 1.5-10 denier. Bicomponent fibers with 1.5-10 denier is convenient to manufacture, the cost is low, and the cooling element made has a large capillary force, which can better absorb and remove the condensate in the mist to form a dry aerosol, which is conducive to the user sensing a lower temperature.

In the present embodiment, the bicomponent fibers 2 is a filament or staple fiber with a sheath 21 and a core 22 having a concentric structure. The sheath 21 is a copolyester of polyethylene terephthalate, and the core 2 is polyethylene terephthalate.

The sheath 21 of bicomponent fibers 2 has a higher melting point, which can withstand higher aerosol temperatures. For example, in some traditional Chinese medicine aerosol emission devices, the temperature of the heating element is as high as 400° C. or above during operation. Polymers with lower melting points such as polypropylene, or poly L-lactic acid can be used as the sheath 21 of bicomponent fibers 2 if the aerosol emission device works at a low temperature. such as atomizing electronic cigarettes or heating non-burning electronic cigarettes.

### <Operating Principle of Cooling Element>

The cooling element 300 according to the present embodiment is used for cooling the aerosol generated in the aerosol emission device. The aerosol generated by the aerosol emission device is properly cooled by the cooling element 300. The aerosol transfers its own heat to the cooling element 300 through heat exchange to reduce the temperature, the temperature of the cooling element 300 rises after absorbing the heat in the aerosol, and the material in the cooling element 300 partially melts after absorbing the heat, so that the cooling element 300 can absorb a large amount of heat in the aerosol, the temperature of the aerosol is significantly reduced.

The cooling element 300 of the present embodiment is made by bonding the bicomponent fibers 2. The sheath 21 and the core 22 of the bicomponent fibers 2 are both polymers. and the polymers can absorb heat when certain phase change occurs, for example, the crystalline area of the polymer is damaged when the polymer is melted, the polymer converts from a solid state to a viscous flow state. This phase changing process requires to absorb a large amount of heat from the outside.

In the aerosol emission device to which the cooling element 300 according to the present embodiment is applied, the temperature of the aerosol generated by the aerosol emission device is higher than the melting point of the sheath of the bicomponent fibers. The high-temperature aerosol flows in from one end of the cooling element 300 and emits out from the other end, and the sheath of the bicomponent fibers of the cooling element 300 melts when contacting the high-temperature aerosol, so as to absorb a large amount of heat in the aerosol, so that the temperature of the high-temperature aerosol is dropped rapidly.

In the present embodiment, the melting point of the core 22 of the bicomponent fibers 2 of the cooling element 300 is higher than the melting point of the sheath 21 by 25° C. or more, the core 22 of the bicomponent fibers 2 that has a high melting point serves as a skeleton, and the melted sheath 21 becomes a viscous flow state and adheres to the core 22, thereby maintaining the integrity of the cooling element 300.

The cooling element 300 is designed according to application requirements, so that the temperature of the aerosol emitting out from the other end of the cooling element 300 can be dropped to below 65° C. to suit the taste of the smoker.

### <Additional Function of Cooling Element>

As the aerosol flows in from one end of the cooling element 300 and emits out from the other end, the temperature of the aerosol gradually drops, and the partially vaporized aerosol agent and moisture are condensed into small droplets. The cooling element 300 made by bonding the bicomponent fibers 2 has a large number of capillary pores, and the capillary pores can absorb the condensate generated when the aerosol is cooled, so that the aerosol becomes dry, which is beneficial for the user to sense a lower temperature. The condensate can absorb part of substances such as phenols and aldehydes, so the capillary pores of the cooling element 300 absorb the condensate while reduce harmful substances in the aerosol, such as phenols and aldehydes. Additives that reduce phenolic substances, such as glycerol acetate, triethyl citrate, low molecular weight ethylene glycol, or mixture of glycerol acetate and cellulose acetate fibers, can be added to the cooling element. Flavoring agents, such as mint, natural flavors or synthetic flavors, etc . . . can also be added to the cooling element, so that the user can inhale aerosols with different flavors.

### The Twelfth Embodiment

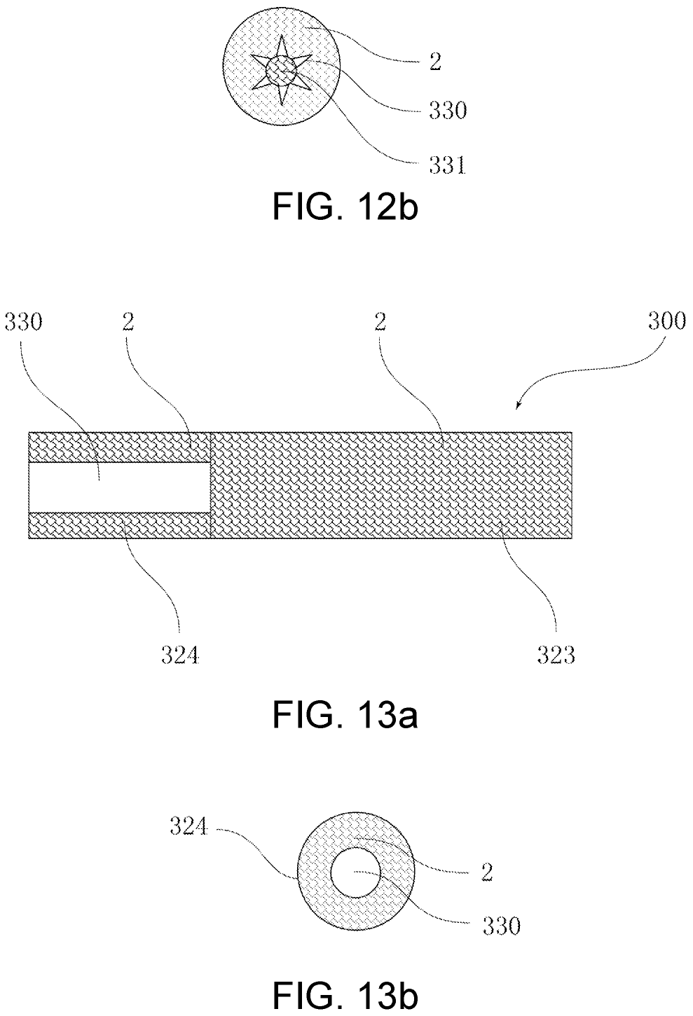

FIG. 12a shows a longitudinal section view of a cooling element according to the twelfth embodiment of the present invention. FIG. 12b shows a cross-sectional view of the cooling element according to the twelfth embodiment of the present invention. The structure of the present embodiment is similar to that of the eleventh embodiment, and the same parts as the eleventh embodiment will not be repeated in the description of this embodiment.

In the present embodiment, the cooling element 300 includes a cooling element through-hole 330 axially penetrating therethrough. The cross section of the cooling element through-hole 330 is configured as a star, as shown in FIG. 2b, an inner core 331 is inserted into the cooling element through-hole 330. The inner core 331 preferably is a cylindrical structure. Since a plurality of airflow channels are formed between the star hollow structure and the cylindrical inner core 331, so that the aerosol can be divided into several small airflows when passing through the cooling element 300, thereby more fully contacting the cooling element 300 and exchanging heat with it.

### The Thirteenth Embodiment

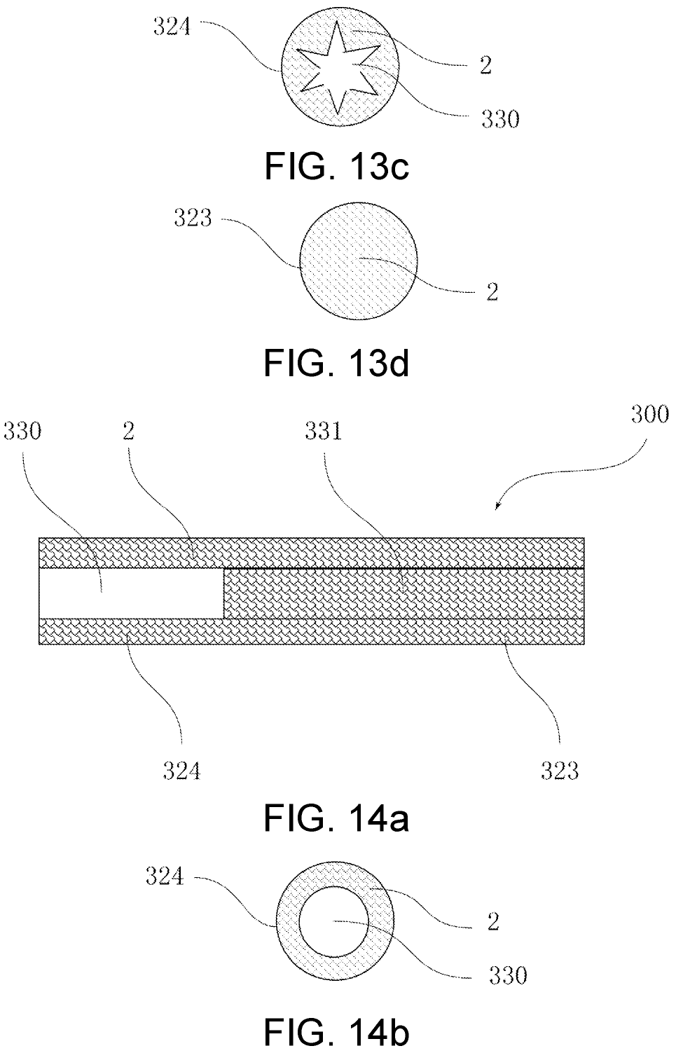

FIG. 13a shows a longitudinal section view of a cooling element according to the thirteenth embodiment of the present invention. FIG. 13b shows a cross-sectional view of a high-temperature cooling section of the cooling element according to the thirteenth embodiment of the present invention. FIG. 13c shows another cross-sectional view of the high-temperature cooling section of the cooling element according to the thirteenth embodiment of the present invention.

FIG. 13d shows a cross-sectional view of a low-temperature cooling section of the cooling element according to the thirteenth embodiment of the present invention. The structure of the present embodiment is similar to that of the eleventh embodiment, and the same parts as the eleventh embodiment will not be repeated in the description of this embodiment.

As shown in FIGS. 13a to 13d, a cooling element 300 includes a high-temperature cooling section 324 and a low-temperature cooling section 323. In an aerosol emission device to which the cooling element 300 according to the present embodiment is applied, an aerosol generated in the aerosol emission device flows in from the end of the high-temperature cooling section 324 of the cooling element 300 and emits out from the end of the low-temperature cooling section 323.

As shown in FIG. 13b, the high-temperature cooling section 324 of the cooling element 300 has a cooling element through-hole 330 axially penetrating through the high-temperature cooling section 324. The high-temperature cooling section 324 preferably is configured as a hollow structure which the cross section of the cooling element through-hole 330 is a circular, and the cross section of the high-temperature cooling section 324 is a circular ring. As shown in FIG. 13d, the high-temperature cooling section 324 of the cooling element 300 may be configured as a hollow structure which the cross section of the cooling element through-hole 330 is a star, and the cross section of the high-temperature cooling section 324 is a star ring, that is, the cross-sectional shape of the inner hole of the hollow structure may be a pentagram, a hexagram, or the like. The high-temperature cooling section 324 adopts the hollow structure, which can reduce the resistance of the aerosol passing through the high-temperature cooling section 324, so that the high-temperature aerosol can pass through the hollow channel with a low air resistance. When the inner surface of the hollow channel is in contact with the high-temperature aerosol, the sheath 21 of the bicomponent fibers 2 absorbs a large amount of heat from the high-temperature aerosol and then melts, so that the temperature of the aerosol is dropped rapidly. When the high-temperature aerosol mainly passes through the hollow channel, the outer periphery of the high-temperature cooling section 324 is far away from the high-temperature aerosol, and the temperature has dropped to a lower temperature when the temperature is transferred to the outer periphery, thereby it can prevent the outer peripheral wall of the high-temperature cooling section 324 from being deformed due to high temperature or prevent the structure and performance of the aerosol emission device from being damaged.

In the present embodiment, the high-temperature cooling section 324 of the cooling element 300 is made by thermally bonding the bicomponent fibers 2, preferably the high-temperature cooling section 324 has a porosity of 80%, the bicomponent fibers 2 are staple fibers and have a sheath 21 and a core 22 which are a concentric structure.

As shown in FIG. 13c, in the present embodiment, the low-temperature cooling section 323 adopts a non-hollow structure, and the cross section of the low-temperature cooling section 323 is a solid circular surface. The low-temperature cooling section 323 has a porosity of 90-95%.

and is made by bonding the bicomponent fibers 2 with an eccentric structure. Although the low-temperature cooling section 323 is the non-hollow structure, since the porosity of the low-temperature cooling section 323 is high, it still has a lower air resistance.

After the aerosol is cooled by the high-temperature cooling section 324, the aerosol with a lower temperature enters the low-temperature cooling section 323. The low-temperature cooling section 323 exchanges heat with the aerosol, the low-temperature cooling section 323 absorbs the heat and the temperature thereof rises, and the aerosol transfers the heat to the low-temperature cooling section 323 and the temperature further drops. If the temperature of the aerosol after cooled by the high-temperature cooling section 324 is higher than the melting point of the sheath 21 of the bicomponent fibers 2 of the low-temperature cooling section 323, the sheath 21 of the bicomponent fibers 2 of the low-temperature cooling section 323 will be partially melted, so that the temperature of the aerosol is dropped rapidly. The cooling element 300 is designed according to the application requirements, so that the temperature of the aerosol emitting out from the end surface of the low-temperature cooling section 323 can be dropped to below 65° C. to suit the taste of the smoker. The low-temperature cooling section 323 with the non-hollow structure is adopted. when the aerosol penetrates the low-temperature cooling section 323, it can more fully exchange heat with the bicomponent fibers 2, and can better reduce the temperature of the aerosol.

In the present embodiment. preferably, the melting point of the sheath 21 of the high-temperature cooling section 324 is greater than that of the sheath 21 of the low-temperature cooling section 323. In the bicomponent fibers 2 of the high-temperature cooling section 324, the sheath 21 is poly-L-lactic acid having a melting point of about 170° C., and the core 22 is polyethylene terephthalate having a melting point of about 265° C. In the bicomponent fibers 2 of the low-temperature cooling section 323, the sheath 21 is poly-D, L-lactic acid having a melting point of about 130° C., and the core 22 is poly-L-lactic acid having a melting point of about 170° C.

If the aerosol emission device to which the cooling element 300 of the present embodiment is applied carries components such as nicotine and glycerin or the like, when the aerosol emission device is heated to about 375° C., the components such as nicotine and glycerin or the like, is volatilized and is emitted out with the generated aerosol along with the user's inhalation, and the high-temperature aerosol enters the high-temperature cooling section 324 of the cooling element 300. The inner wall of the hollow channel of the high-temperature cooling section 324 is in contact with the high-temperature aerosol and exchanges heat with that, the sheath 21 of a part of the bicomponent fibers 2 is melted when contacting the high-temperature aerosol, meanwhile absorbs a large amount of heat in the aerosol, so that the temperature of the high-temperature aerosol is dropped rapidly, and a part of the glycerin condenses into liquid which is absorbed by the high-temperature cooling section 324. The high-melting point core 22 of the bicomponent fibers 2 of the high-temperature cooling section 324 acts as a skeleton, and the melted sheath 21 becomes viscous flow state and adheres to the core 22, thereby maintaining the integrity of the cooling element 300.

After being cooled by the high-temperature cooling section, the aerosol with a lower temperature enters the low-temperature cooling section 323 of the cooling element 300. If the temperature of the aerosol entering the low-temperature cooling section 323 is still higher than 130° C. the sheath 21 of the bicomponent fibers 2 of the low-temperature cooling section 323 will be partially melted, so that the temperature of the aerosol is rapidly dropped to below 130° C. Subsequently, the low-temperature cooling section 323 continues to exchange heat with the aerosol, and absorbs heat by utilizing the phase change between 55° C. and 70° C. of polylactic acid in the low-temperature cooling section 323, so that the temperature of the aerosol is further dropped to suit the taste of the smoker.

As shown in FIG. 13d, in the present embodiment, the low-temperature cooling section 323 adopts a non-hollow structure, when the aerosol penetrates the low-temperature cooling section 323, the aerosol is fully in contact with the bicomponent fibers and exchange heat with that, so that the temperature of the aerosol emitting out from the end of the low-temperature cooling section 323 is dropped to below 65° C. Part of the glycerin and moisture in the aerosol is absorbed by the low-temperature cooling section 323 after being condensed into a liquid in the low-temperature cooling section 323, so that the aerosol becomes dry, which is beneficial for the user to sense a lower temperature.

Due to the fact that the condensate can dissolve part of substances such as aldehydes and phenols, after the condensate is absorbed by the capillary pores in the cooling element 300, the inhalation of harmful substances with aldehyde and phenol by the user can be reduced, 1-3% of glycerol acetate or a mixture of glycerol acetate and cellulose acetate fibers is added to the low-temperature cooling section 323 of the cooling element 300 of the present embodiment to reduce the content of phenolic substances in the aerosol.

### The Fourteenth Embodiment

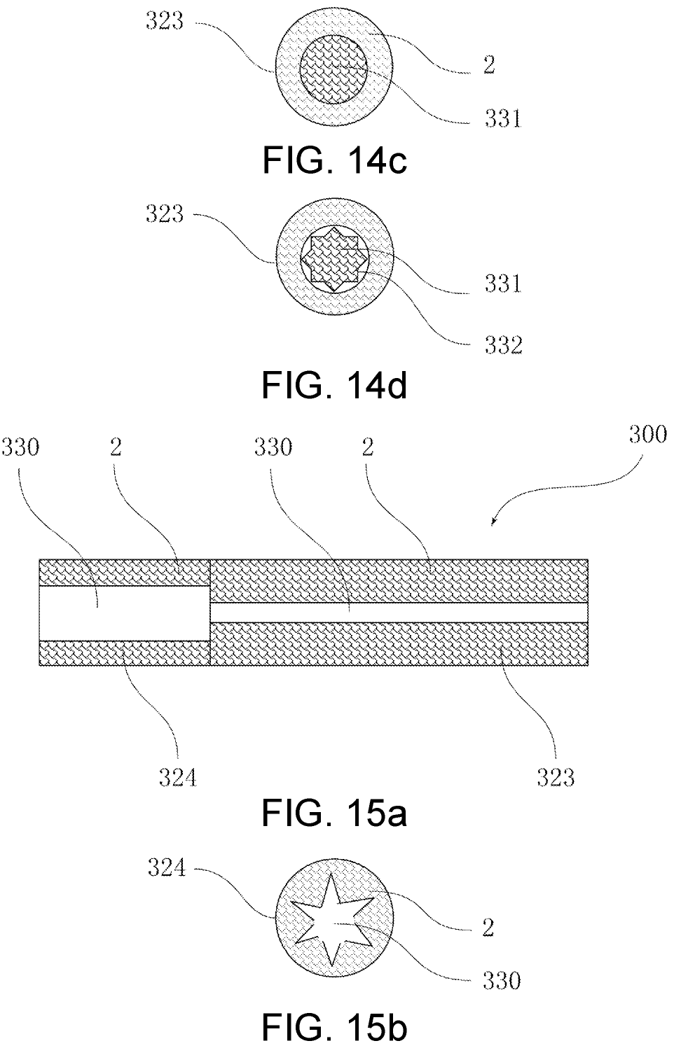

FIG. 14a shows a longitudinal section view of a cooling element according to the fourteenth embodiment of the present invention. FIG. 14b shows a cross-sectional view of a high-temperature cooling section of the cooling element according to the fourteenth embodiment of the present invention. FIG. 14c shows a cross-sectional view of a low-temperature cooling section of the cooling element according to the fourteenth embodiment of the present invention. FIG. 14d shows another cross-sectional view of the low-temperature cooling section of the cooling element according to the thirteenth embodiment of the present invention. The structure of the present embodiment is similar to that of the eleventh embodiment, and the same parts as the eleventh embodiment will not be repeated in the description of this embodiment.

In the present embodiment, a cooling element 300 includes a high-temperature cooling section 324 and a low-temperature cooling section 323. The high-temperature cooling section 324 has a cooling element through-hole 330 axially penetrating therethrough. Preferably the high-temperature cooling section 324 is configured as a hollow structure which the cross section of the cooling element through-hole 330 is a circular, and the cross section of the high-temperature cooling section 324 is a circular ring.

As shown in FIG. 14c, the low-temperature cooling section 323 has a cooling element through-hole 330 axially penetrating therethrough. The cross section of the cooling element through-hole 330 of the low-temperature cooling section 323 is a circular, an inner core 331 is inserted into the cooling element through-hole 330 of the low-temperature cooling section 323 and preferably has a cylindrical structure.

As shown in FIG. 14d, grooves 332 may be provided on the surface of the inner core 331 of the low-temperature cooling section 323 to reduce an air resistance.

In the present embodiment, the cooling element 300 and the inner core 331 are made by bonding bicomponent fibers 2. A sheath 21 of the bicomponent fibers 2 is polylactic acid having a melting point of about 120° C., and a core 22 is polylactic acid having a melting point of about 160° C.

In order to save the cost, the sheath 21 can be replaced by polyethylene, polyolefin or copolyester having a melting point of 100-120° C., and the core 22 can be replaced by polypropylene or polyethylene terephthalate.

The porosities of the high-temperature cooling section 324 and the low-temperature cooling section 323 preferably are 85%, and the porosities of the inner core 331 of the low-temperature cooling section 323 preferably are 85-95%.

### The Fifteenth Embodiment

FIG. 15a shows a longitudinal section view of a cooling element according to the fifteenth embodiment of the present invention. FIG. 15b shows a cross-sectional view of a high-temperature cooling section of the cooling element according to the fifteenth embodiment of the present invention. FIG. 15c shows a cross-sectional view of a low-temperature cooling section of the cooling element according to the fifteenth embodiment of the present invention. The structure of the present embodiment is similar to that of the eleventh embodiment, and the same parts as the eleventh embodiment will not be repeated in the description of this embodiment.

In the present embodiment, a cooling element 300 includes a high-temperature cooling section 324 and a low-temperature cooling section 323. As shown in FIG. 15b, the high-temperature cooling section 324 has a cooling element through-hole 330 axially penetrating therethrough. Preferably the high-temperature cooling section 324 is configured as a hollow structure which the cross section of the cooling element through-hole 330 is a star, and the cross section of the high-temperature cooling section 324 is a star ring.

As shown in FIG. 15c, the low-temperature cooling section 323 also has a cooling element through-hole 330 axially penetrating therethrough. Preferably the low-temperature cooling section 323 is configured as a hollow structure which the cross section of the cooling element through-hole 330 is a star, and the cross section of the low-temperature cooling section 323 is a star ring. The cross-sectional area of the cooling element through-hole 330 of the high-temperature cooling section 324 is larger than that of the cooling element through-hole 330 of the low-temperature cooling section 323.

In the present embodiment, the cooling element 300 is made by bonding bicomponent fibers 2. A sheath 21 of the bicomponent fibers 2 is polylactic acid having a melting point of about 130° C., and a core 22 is polylactic acid having a melting point of about 170° C. In order to save the cost, the sheath 21 can be replaced by polyethylene, polyolefin or copolyester having a melting point of 100-130° C. and the core 22 can be replaced by polypropylene or polyethylene terephthalate.

The porosity of the high-temperature cooling section 324 preferably is 75-85%, and the porosity of the low-temperature cooling section 323 preferably is 85-90%.

### The Sixteenth Embodiment

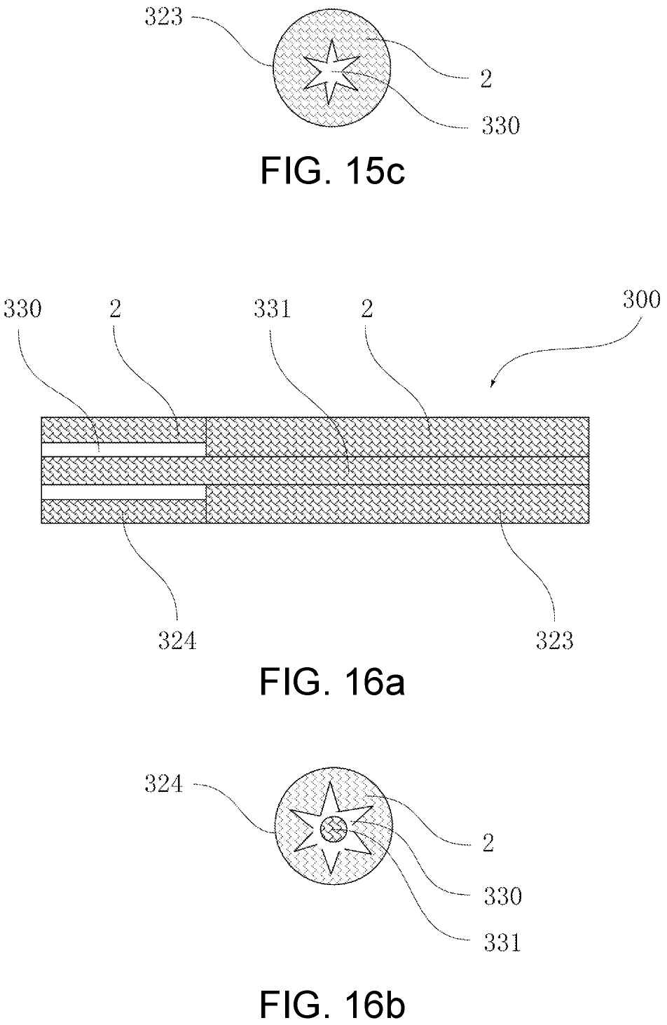

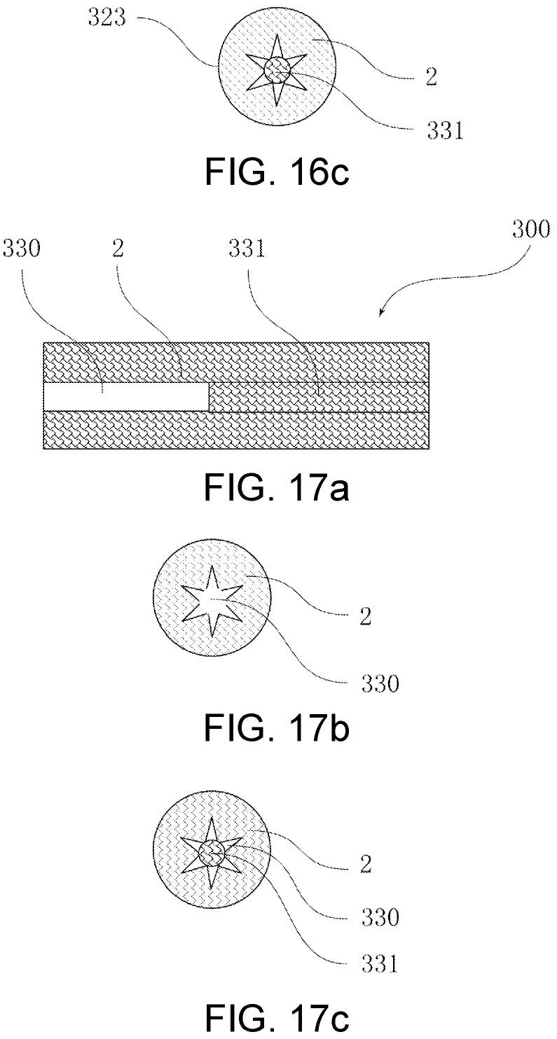

FIG. 16a shows a longitudinal section view of a cooling element according to the sixteenth embodiment of the present invention. FIG. 16b shows a cross-sectional view of a high-temperature cooling section of the cooling element according to the sixteenth embodiment of the present invention. FIG. 16c shows a cross-sectional view of a low-temperature cooling section of the cooling element according to the sixteenth embodiment of the present invention. The structure of the present embodiment is similar to that of the eleventh embodiment, and the same parts as the eleventh embodiment will not be repeated in the description of this embodiment.

In the present embodiment, the cooling element 300 includes a high-temperature cooling section 324 and a low-temperature cooling section 323. As shown in FIG. 16b, the high-temperature cooling section 324 has a cooling element through-hole 330 axially penetrating therethrough. Preferably the high-temperature cooling section 324 is configured as a hollow structure which the cross section of the cooling element through-hole 330 is a star, and the cross section of the high-temperature cooling section 324 is a star ring.

As shown in FIG. 16c, the low-temperature cooling section 323 also has a cooling element through-hole 330 axially penetrating therethrough. Preferably the low-temperature cooling section 323 is configured as a hollow structure which the cross section of the cooling element through-hole 330 is a star, and the cross section of the low-temperature cooling section 323 is a star ring. The cross-sectional area of the cooling element through-hole 330 of the high-temperature cooling section 324 is larger than that of the cooling element through-hole 330 of the low-temperature cooling section 323.

As shown in FIGS. 16a to 16c, in order to improve the cooling effect, an inner core 331 can be inserted into the cooling element through-holes 330 of the high-temperature cooling section 324 and the low-temperature cooling section 323, and the diameter of the inner core 331 is not larger than the inner diameter of the cooling element through-hole 330 of the low-temperature cooling section 323.

### The Seventeenth Embodiment

FIG. 17a shows a longitudinal section view of a cooling element according to the seventeenth embodiment of the present invention. FIG. 17b shows a cross-sectional view of a high-temperature cooling section of the cooling element according to the seventeenth embodiment of the present invention. FIG. 17c shows a cross-sectional view of a low-temperature cooling section of the cooling element according to the seventeenth embodiment of the present invention. The structure of the present embodiment is similar to that of the eleventh embodiment, and the same parts as the eleventh embodiment will not be repeated in the description of this embodiment.

The high-temperature cooling section 324 has a cooling element through-hole 330. Preferably the high-temperature cooling section 324 is configured as a hollow structure which the cross section of the cooling element through-hole 330 is a star, and the cross section of the high-temperature cooling section 324 is a star ring.

As shown in FIG. 17c, the low-temperature cooling section 323 also has a cooling element through-hole 330 axially penetrating therethrough. Preferably the low-temperature cooling section 323 is configured as a hollow structure which the cross section of the cooling element through-hole 330 is a star, and the cross section of the low-temperature cooling section 324 is a star ring. As shown in FIGS. 17a to 17c, an inner core 331 is inserted into the cooling element through-hole 330 of the low-temperature cooling section 323 and is loaded with flavoring agents, such as mint, essence, or the like.

In the present embodiment, the high-temperature cooling section 324 and the low-temperature cooling section 323 may be integrally formed, and the porosities preferably are 85%. In the present embodiment, the cooling element 300 is made by bonding bicomponent fibers 2. A sheath 21 of the bicomponent fibers 2 is polylactic acid having a melting point of about 170° C. and a core 22 is polyethylene terephthalate having a melting point of about 265° C., in order to reduce the cost, the sheath 21 can be replaced by polypropylene.

### The Eighteenth Embodiment

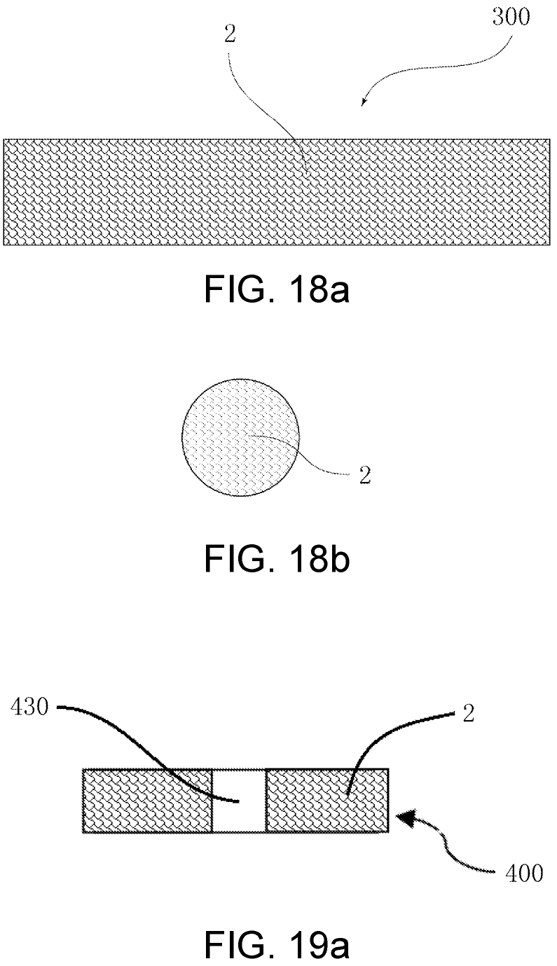

FIG. 18a shows a longitudinal section view of a cooling element according to the eighteenth embodiment of the present invention. FIG. 18b shows a cross-sectional view of the cooling element according to the eighteenth embodiment of the present invention. The structure of the present embodiment is similar to that of the eleventh embodiment, and the same parts as the eleventh embodiment will not be repeated in the description of this embodiment.

In the present embodiment, a cooling element 300 is made by bonding the bicomponent fibers 2, and the porosity of that is 90%. The bicomponent fibers 2 are staple fibers and have a sheath 21 and a core 22 which are a concentric or eccentric structure, the sheath 21 is polylactic acid having a melting point of 125-135° C., and the core 22 is polylactic acid having a melting point of 160-185° C.

In the present embodiment, the cooling element 300 has a non-hollow structure, and the aerosol can fully contact the cooling element 300 and exchange heat with the cooling element 300 when passing through the cooling element 300. In order to save the cost, the sheath 21 can be replaced by polyethylene, polypropylene, etc., and the core 22 can be replaced by polypropylene. polyethylene terephthalate, etc.

To sum up ' the present invention relates to a cooling element 300, the cooling element 300 is made by bonding bicomponent fibers 2, and the bicomponent fibers 2 have a sheath 21 and a core 22. The cooling element 300 made by bonding the bicomponent fibers 2 has a large number of capillary pores, which has a good absorption effect on the condensate generated when an aerosol is cooled, so that the aerosol becomes dry, which is beneficial for the user to sense lower temperature. The cooling element 300 made by bonding the bicomponent fibers 2 can be made into a hollow structure and a non-hollow structure, which can be used alone or in combination according to the needs, so as to achieve an appropriate cooling effect and air resistance. The cooling element 300 of the present invention can be applied to various aerosol emission devices. such as an aerosol emission device containing essence, nicotine, or vaporizable Chinese herbal medicinal component, etc. The foregoing embodiments of the present invention are only intended to illustrate the principle and advantages of the present invention rather than limiting the present invention. For example, the cooling element 300 may be made by mixing two different bicomponent fibers, or some monocomponent fibers are mixed with the bicomponent fibers in order to reduce the cost without affecting the overall performance of the cooling element 300.

### The Nineteenth Embodiment

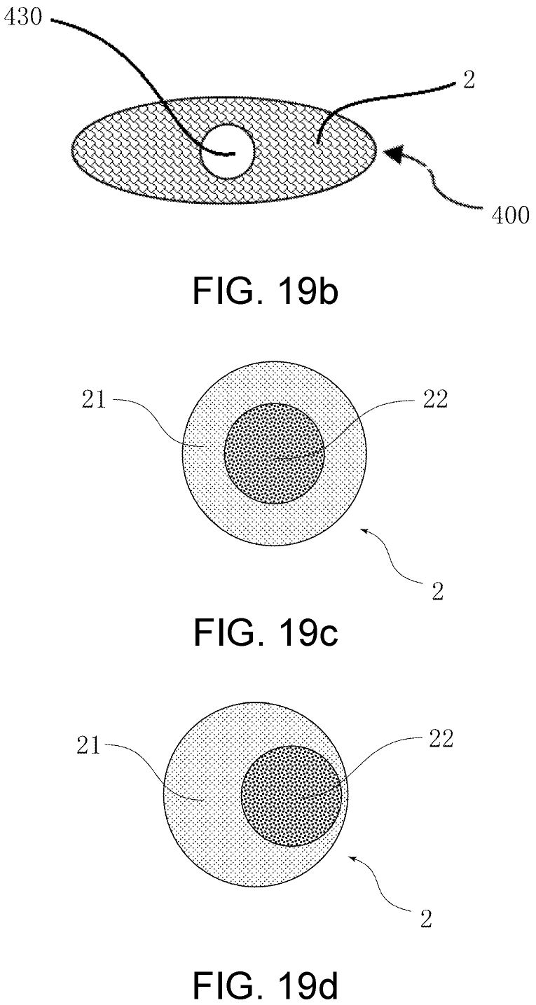

FIG. 19a shows a longitudinal section view of a condensate absorbing element according to the nineteenth embodiment disclosed in the present invention. FIG. 19b shows a cross-sectional view of the condensate absorbing element according to the nineteenth embodiment of the present invention.

As shown in FIGS. 19a and 19b, a condensate absorbing element 400 according to the nineteenth embodiment of the present invention is used for absorbing condensate in an aerosol channel of an aerosol emission device. The condensate absorbing element 400 has a three-dimensional network structure which is formed by thermally bonding bicomponent filaments 2 with a concentric structure, and the bicomponent fibers 2 have a sheath 21 and a core 22.

The sheath 21 preferably is polylactic acid, or, preferably is polyester such as polyethylene, polypropylene, PBT or PTT, etc., or a low melting point copolyester such as polyethylene terephthalate.

The polylactic acid preferably is poly-D-lactic acid, poly-L-lactic acid or poly-D, L-lactic acid.

### <Density and Rigidity of the Condensate Absorbing Element>

The condensate absorbing element 400 of the present embodiment has a density of 0.1 g/cm³ to 0.4 g/cm³, preferably 0.2 g/cm³ to 0.3 g/cm³. When the condensate absorbing element 400 has a density less than 0.1 g/cm³, the capillary force of the condensate absorbing element 400 is small, the ability of absorbing the condensate is poor, and the axial rigidity of the condensate absorbing element 400 is too small, which is unfavorable for assembly in the aerosol emission device, especially is unfavorable for high-speed automated assembly; when the condensate absorbing element 400 has a density greater than 0.4 g/cm³, the radial rigidity of the condensate absorbing element 400 is too high, it is difficult to be assembled in the aerosol emission device, and the liquid absorbent capacity of the condensate absorbing element 400 per unit volume is too small, and the space utilization efficiency is poor, which is unfavorable for using in an aerosol emission device with a narrow space.

The rigidity comparison method herein is: placing the condensate absorbing element 400 in the axial or radial direction, clamping it between two parallel plates, then measuring an axial height or a radial height of the condensate absorbing element 400 before compression; under the condition of applying the same acting force, measuring an axial height or a radial height after the two plates axially or radially compress the condensate absorbing element 400, and calculating a deformation amount of the compression which is the difference between the axial height or the radial height before compression and the axial height or the radial height after compression; and dividing the deformation amount of the compression by the axial height or the radial height of the condensate absorbing element 400 before compression to obtain a compression ratio. The smaller the compression ratio, the higher the rigidity, and the greater the compression ratio, the less the rigidity.

The axial rigidity of the condensate absorbing element 400 is greater than its radial rigidity. During the preparing process of the condensate absorbing element 400, the preparing process can be controlled so that the axial rigidity of the condensate absorbing element 400 is greater than its radial rigidity. Therefore, during assembly, the condensate absorbing element 400 in the radial direction can be self-adaptively deformed under the action of the axial force of the condensate absorbing element 400, and self-adaptively fixed in the aerosol emitting device, which facilitates high-speed automatic assembly.

### <Bicomponent Fibers>

FIG. 19c shows an enlarged cross-sectional schematic view of bicomponent fibers of FIGS. 19a and 19b. As shown in FIG. 19c, the sheath 21 and the core 22 are concentric structure. The bicomponent fibers 2 with the concentric structure have a greater rigidity are easy to produce.

FIG. 19d shows another enlarged cross-sectional schematic view of the bicomponent fibers of FIGS. 19a and 19b. As shown in FIG. 19d, the sheath 21 and the core 22 are eccentric structure. The bicomponent fibers 2 with an eccentric structure are relatively soft and fluffy, and it is easy to manufacture the condensate absorbing element 400 with a lower density.

The bicomponent fibers 2 are filaments or staple fibers. The condensate absorbing element 400 made from filaments has a higher strength, and the condensate absorbing element 400 made from short fibers has a better elasticity. The manufacturer can select suitable bicomponent fibers to make the condensate absorbing element 400 with a suitable density and suitable shape according to the performance requirements of the condensate absorbing element 400.

The core 22 of the bicomponent fibers 2 has a higher melting point than the sheath 21 by 20° C. or more. The condensate absorbing element 400 of the present embodiment is made by thermally bonding the bicomponent fibers 2 with a sheath-and-core structure, and the core 22 of the bicomponent fibers 2 has a higher melting point than the sheath 21 by 20° C. or more, so that the core 22 can maintain a certain rigidity when thermal bonding is performed between the fibers. which is convenient to manufacture the condensate absorbing element 400.

The sheath 21 of bicomponent fibers 2 is polylactic acid, abbreviated as PLA. Polylactic acid is made from lactic acid through chemical reactions, and there are optical isomers of L and D. Polylactic acid includes poly L-lactic acid, poly D-lactic acid, and poly D, L-lactic acid. Different polylactic acids have different melting points. Due to differences in raw material purity and production processes, the same type of polylactic acid from different manufacturers may have the same or different melting points; Polylactic acid of the same type but different models from the same manufacturer may have the same or different melting points. The material of suitable core 22 can be selected based on the melting point of polylactic acid in the sheath 21. For example, the core 22 can be polypropylene, polyethylene terephthalate, etc. if the sheath 21 adopts poly D, L-lactic with a melting point of 125 to 135° C., or, the core 22 can be poly D-lactic acid or poly L-lactic acid with a melting point of 165 to 180° C. Another example, the core 22 can polyethylene terephthalate, polybutylene terephthalate, propylene terephthalate, polyamide, etc. if the sheath 21 adopts poly D-lactic acid or poly L-lactic acid with a melting point of 155-170° C. Polylactic acid is a biodegradable material that can be completely decomposed by microorganisms into carbon dioxide and water. Especially the condensate absorbing element can be completely decomposed by microorganisms when both the sheath 21 and the core 22 are polylactic acid, which can greatly reduce the environmental pollution caused by the abandonment of the condensate absorbing element 400 after use.

The sheath 21 of the bicomponent fibers 2 also can be polyethylene, polypropylene, copolyesters of polytrimethylene terephthalate, polybutylene terephthalate, or polyethylene terephthalate, or polyamide-6. The material of the core 22 can be selected based on the melting point of the sheath 21. For example, the polypropylene. polyethylene terephthalate, and other materials can be used as the core 22 if the high-density polyethylene with a melting point of 125-135° C. is used as the sheath 21. Another example, the polyethylene terephthalate, polyamide, etc. can be selected as the core 22 if the polypropylene with a melting point of 160-170° C. is used as the sheath 21. Another example, the polyethylene terephthalate, etc. can be selected as the core 22 if the low melting point copolyesters with a melting point of 110-120° C. is used as the sheath 21. Another example, the polyethylene terephthalate with a melting point of 255-265° C. can be used as the core 22 if the polybutylene terephthalate or polytrimethylene terephthalate with a melting point of 225-235° C. is used as the sheath 21.

Two component fiber 2 is bonded to form a three-dimensional network structure. There are various methods for bonding, such as using glue (the most common bonding method), plasticizers (the bonding method for cigarette filter tips), and so on. The present invention preferably forms a three-dimensional network structure through thermal bonding. The cost is low and impurities are not introduced by using thermal bonding method.

### <Fineness of Bicomponent Fibers>

The bicomponent fibers 2 from which the condensate absorbing element 400 of the present invention is made have a fineness of 1-10 denier, preferably 2-6 denier. The bicomponent fibers 2 with the sheath-and-core structure which has a fineness less than 1 denier is difficult to manufacture and has high cost. The condensate absorbing element 400 made from fibers with a fineness higher than 10 denier has insufficient capillary force and poor ability to absorb condensate.

The bicomponent fibers 2 which has the sheath-and-core structure and has 1-10 denier is easily thermally bonded into the condensate absorbing element 400 which has a lower density and has the three-dimensional structure with a suitable capillary force, and the bicomponent fibers 2 with the sheath-and-core structure having 2-6 denier is particularly suitable and has lower cost.

### <Shape of Condensate Absorbing Element>

The condensate absorbing element 400 of the present embodiment can be made into a suitable cross-sectional shape according to the internal structure needs of the aerosol emission device, such as a circular, an elliptical, a rectangular, or a combination of various geometric shapes, so that the condensate absorption element 400 can be conveniently assembled in the aerosol emission device. The condensate absorbing element through-hole 430 can be provided in the axial direction of the condensate absorbing element 400 according to the requirements. When the aerosol passes through the periphery of the condensate absorbing element 400 or through the condensate absorbing element through-hole 430, the condensate around the aerosol is in contact with the condensate absorbing element to be absorbed. The condensate absorbing element through-hole 430 may be a circular, an elliptical, a rectangular or a combination of various geometric shapes, and the number of that may be one or more.

In the present embodiment, preferably the condensate absorbing element 400 has a three-dimensional network structure formed by thermally bonding bicomponent fibers 2 with a concentric structure. The bicomponent fibers 2 have a fineness of 2 denier, the sheath 21 is poly-D, L-lactic acid having a melting point of 125-135° C., and the core 22 is poly-L-lactic acid having a melting point of 165-180° C., the prepared condensate absorbing element 400 has a density of 0.2 g/cm³ to 0.3 g/cm³, the condensate absorbing element 400 has characteristics in that the liquid absorbent capacity is large and the absorption rate is fast. As shown in FIG. 1b, in the present embodiment, the condensate absorbing element 400 has an elliptical of the cross-sectional shape, and is provided with an axial condensate absorbing element through-hole 430 having a circular cross section. When the aerosol passes through the axial condensate absorbing element through-hole 430 of the condensate absorbing element 400, the condensate around the aerosol is in contact with the peripheral wall of the axial condensate absorbing element through-hole 430 to be absorbed, which preventing the condensate from being sucked into the mouth by the user and improving the user's experience.

In the present embodiment, the sheath 21 may also be high-density polyethylene having a melting point of 125-135° C., and the core 22 may be polypropylene having a melting point of 160-170° C.

### The Twentieth Embodiment

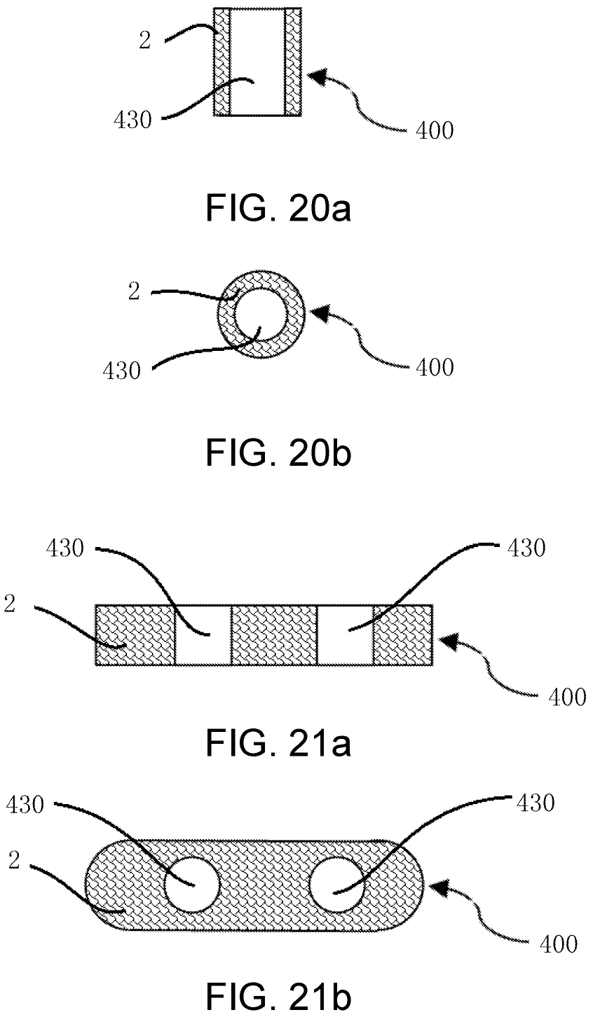

FIG. 20a shows a longitudinal section view of a condensate absorbing according to the twentieth embodiment of the present invention. FIG. 20b shows a cross-sectional view of the condensate absorbing according to the twentieth embodiment of the present invention. The structure of the present embodiment is similar to that of the nineteenth embodiment, and the same parts as the nineteenth embodiment will not be repeated in the description of this embodiment.

In the present embodiment, a condensate absorbing element 400 has a three-dimensional network structure formed by thermally bonding bicomponent fibers 2 with a concentric structure.

The bicomponent fibers 2 are filaments and have a fineness of 4 denier. The sheath 21 of the bicomponent fibers 2 is poly-L-lactic acid or poly-D-lactic acid having a melting point of 165-180° C., and the core 22 is polyethylene terephthalate having a melting point of 255-265° C. The condensate absorbing element 400 has a better temperature resistance, and can be installed at a part of the aerosol channel that close to the atomizer. Preferably the prepared condensate absorbing element 400 has a density of 0.25 g/cm³, which has a relatively high rigidity and is suitable for high-speed automated assembly. As shown in FIG. 20b, in the present embodiment, the cross-sectional profile of the condensate absorbing element 400 is a circular, and so does the axial condensate absorbing element through-hole 430.

If the condensate absorbing element of the present embodiment is used in a part that farther away from the atomizer, the sheath can be replaced by polylactic acid having a lower melting point, such as poly-L-lactic acid or poly-D-lactic acid having a melting point of 145-160° C. poly-L-lactic acid or poly-D-lactic acid having a melting point of 155-170° C. or poly-D, L-lactic acid having a melting point of 125-135° C. etc.

In the present embodiment, also, the sheath 21 of the bicomponent fibers 2 may be polybutylene terephthalate having a melting point of 225-235° C. and the core 22 may be polyethylene terephthalate having a melting point of 255-265° C. If the condensate absorbing element of the present embodiment is used in a part that farther away from the atomizer, the sheath may be polymer having a lower melting point, such as polypropylene having a melting point of 160-170° C.

### The Twenty-First Embodiment

FIG. 21a shows a longitudinal section view of a condensate absorbing according to the twenty-first embodiment of the present invention. FIG. 21b shows a cross-sectional view of the condensate absorbing according to the twenty-first embodiment of the present invention. The structure of the present embodiment is similar to that of the nineteenth embodiment, and the same parts as the nineteenth embodiment will not be repeated in the description of this embodiment.

In the present embodiment, a condensate absorbing element 400 has a three-dimensional network structure formed by thermally bonding bicomponent fibers 2 with an eccentric structure. The bicomponent fibers 2 have a fineness of 10 denier. The sheath 21 is poly-D, L-lactic acid having a melting point of 125-135° C., and the core 22 is poly-L-lactic acid having a melting point of about 165-180° C. As shown in FIG. 21b, in the present embodiment, the cross section of the condensate absorbing element 400 is composed of a rectangle and two semicircles, and the condensate absorbing element 400 is provided with two axial condensate absorbing element through-holes 430, which is particularly suitable for the aerosol emission device designed with two aerosol channels at a mouthpiece portion.

In the present embodiment, also, the sheath 21 may be low melting point copolyester having a melting point of 110-120° C., and the core 22 may be polyethylene terephthalate having a melting point of about 255-265° C.

### The Twenty-Second Embodiment

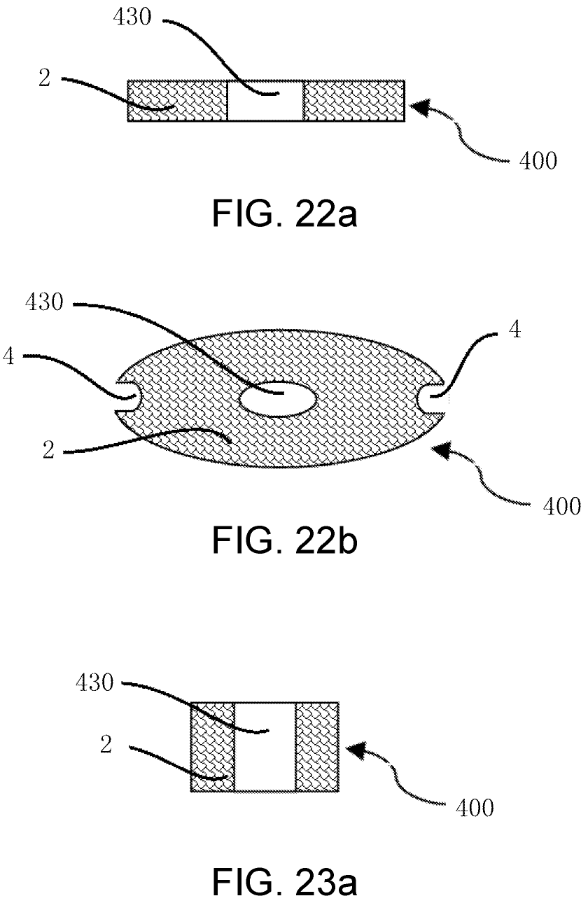

FIG. 22a shows a longitudinal section view of a condensate absorbing according to the twenty-second embodiment of the present invention. FIG. 22b shows a cross-sectional view of the condensate absorbing according to the twenty-second embodiment of the present invention. The structure of the present embodiment is similar to that of the nineteenth embodiment, and the same parts as the nineteenth embodiment will not be repeated in the description of this embodiment.

In the present embodiment, a condensate absorbing element 400 has a three-dimensional network structure formed by thermally bonding bicomponent fibers 2 with an eccentric structure. The bicomponent fibers 2 are staple fibers and have a fineness of 6 denier. The sheath 21 is poly D, L lactic acid having a melting point of 115-125° C., and the core 22 is poly L-lactic acid having a melting point of 155-170° C. The condensate absorbing element made by the bicomponent fibers 2 with the eccentric structure has a better elasticity in the radial direction, which is convenient to be installed and fixed in the aerosol emission device. The condensate absorbing element 400 has a density of 0.1 g/cm³ to 0.2 g/cm³, the lower density enables the unit volume of the condensate absorbing element 400 to have a larger liquid absorbent capacity. As shown in FIG. 22b, in the present embodiment, the condensate absorbing element 400 is provided with grooves 4 at both ends of the long axis, so that it is convenient to be installed and fixed in an elliptical space. In the present embodiment, the core 22 can be replaced by polypropylene or polyethylene terephthalate to reduce the cost.

In the present embodiment, also, the sheath 21 may be low density polyethylene having a melting point of 110-125° C., and the core 22 may be polypropylene having a melting point of 160° C. to 170° C.

### The Twenty-Third Embodiment

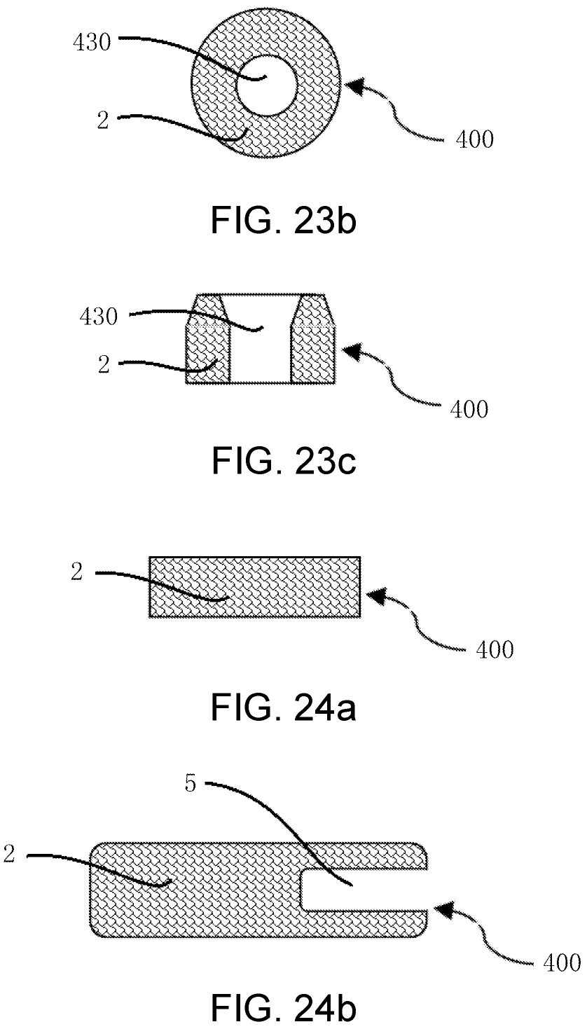

FIG. 23a shows a longitudinal section view of a condensate absorbing element according to the twenty-third embodiment disclosed in the present invention before installation, wherein the upper and lower direction is axial direction and is the direction that force is applied during installation; FIG. 23b shows a cross-sectional view of the condensate absorbing element according to the twenty-third embodiment disclosed in the present invention; FIG. 23c shows a longitudinal section view of the condensate absorbing element according to the twenty-third embodiment disclosed in the present invention after installation. The structure of the present embodiment is similar to that of the nineteenth embodiment, and the same parts as the nineteenth embodiment will not be repeated in the description of this embodiment.

In the present embodiment, a condensate absorbing element 400 has a three-dimensional network structure formed by thermally bonding bicomponent fibers 2. The bicomponent fibers 2 are staple fibers and have a fineness of 1 denier. The sheath 21 is amorphous poly D, L lactic acid without a melting point, and the core 22 is polylactic acid having a melting point of about 160-180° C., the prepared condensate absorbing element 400 has a density of 0.1 g/cm³. The condensate absorbing element 400 is axially inserted into a partially trapezoidal cavity inside the mouthpiece, and the radial direction of the condensate absorbing element 400 is self-adaptively deformed according to the cavity inside the mouthpiece, the longitudinal section view of the condensate absorbing element 400 after assembly is shown in FIG. 23c. By utilizing the characteristic that the condensate absorbing element 400 can self-adaptively deform in the radial direction, the internal space of the aerosol emission device can be fully utilized under the condition of simplifying the design of the condensing absorbing element 400, which is beneficial to the innovative design of the aerosol emission device.

In the present embodiment, also, the sheath 21 may be polyethylene, and the core 22 may be polypropylene.

### The Twenty-Fourth Embodiment

FIG. 24a shows a longitudinal section view of a condensate absorbing according to the twenty-fourth embodiment of the present invention. FIG. 24b shows a cross-sectional view of the condensate absorbing according to the twenty-fourth embodiment of the present invention. The structure of the present embodiment is similar to that of the nineteenth embodiment, and the same parts as the nineteenth embodiment will not be repeated in the description of this embodiment.

In the present embodiment, a condensate absorbing element 400 has a three-dimensional network structure formed by thermally bonding bicomponent fibers 2. The bicomponent fibers 2 have a fineness of 3 denier. The sheath 21 is polylactic acid having a melting point of 125-135° C. and the core 22 is polylactic acid having a melting point of about 165-180° C., the prepared condensate absorbing element 400 has a density of 0.25 g/cm³ to 0.35 g/cm³. As shown in FIG. 24b, one side of the condensate absorbing element 400 of the present embodiment is provided with a groove 5 for conducting aerosol, which is suitable for an aerosol emission device of which an aerosol channel is provided on one side.

In the present embodiment, also, the sheath 21 may be polyethylene having a melting point of 125-135° C., and the core 22 may be polyethylene terephthalate having a melting point of 255-265° C.

The condensate absorbing element 400 of the present invention has a lower density and a higher porosity, and has a large liquid absorbent capacity per unit volume, which is suitable for a compact space of the aerosol emission device. Based on the three-dimensional network structure made by bonding the bicomponent fibers with the sheath-and-core structure, which will not expand or deform after absorbing the condensate, so that the aerosol channel has a stable airflow resistance, which is beneficial to remain the air resistance stable during the using the aerosol emission device, improves user experience.

The condensate absorbing element of the present invention can be customized according to the structure of the aerosol emission device, so as to be conveniently assembled in a precise aerosol emission device. The preparing process can be controlled so that the condensate absorbing element has a greater rigidity in the axial direction than in the radial direction, which facilitates using the axial force when assembling, and facilitates high-speed automatic assembly.

The condensate absorbing element 400 can quickly absorb the condensate while contacting the aerosol, thereby effectively improving the taste.

The foregoing embodiments of the present invention are only intended to illustrate the principle and advantages of the present invention rather than limiting the present invention. For example, two types of bicomponent fibers with different fineness can be mixed to make a condensate absorbing element, or some monocomponent can be mixed into the bicomponent fibers to reduce costs without affecting the overall performance of the condensate absorbing element. Those skilled in the art can make modifications or changes to the foregoing embodiments without departing from the spirit and scope of the present invention. Therefore, all equivalent modifications or changes made by those skilled in the art without departing from the spirit and technical concepts disclosed by the present invention shall still be covered by the claims of the present invention.

### The Twenty-Fifth Embodiment

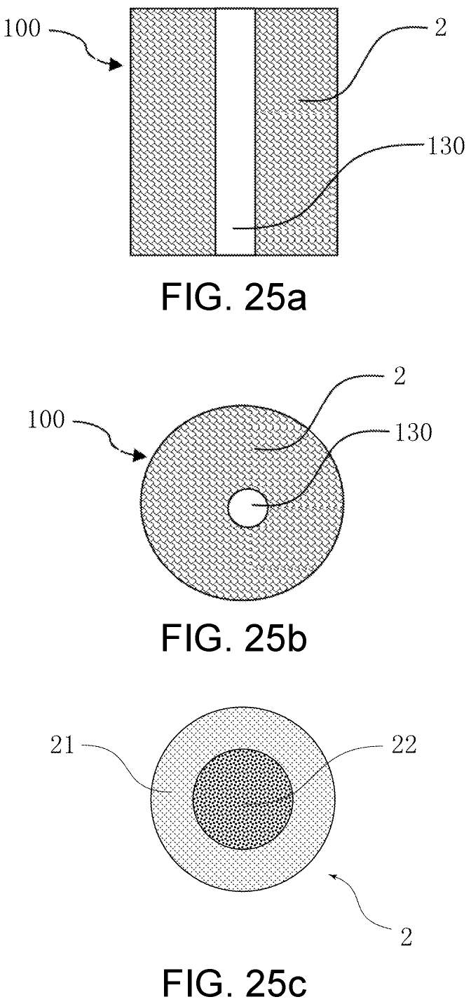

FIG. 25a shows a longitudinal section view of a supporting element according to the twenty-fifth embodiment of the present invention. FIG. 25b shows a cross-sectional view of the supporting element according to the twenty-fifth embodiment of the present invention.

As shown in FIGS. 25a and 25b, a supporting element according to the twenty-fifth embodiment of the present invention is used to support a flavor changing member in an aerosol emission device, and the supporting element 500 has a three-dimensional network structure formed by thermally bonding bicomponent fibers, the bicomponent fibers 2 have a sheath 21 and a core 22.

### <Shape of Supporting Element>

The supporting element 500 may have a supporting element through-hole 530 axially penetrating therethrough. The supporting element through-hole 530 may be used to support a flavor changing member (not shown). For example, the flavor changing member may be a bead which encapsulates flavoring agents such as mint, natural or synthetic flavors. During assembly, the bead is inserted into and supported by the supporting element through-hole 530. When the user using, the bead is broken following the pressure exerted by the user on the supporting element 500, the flavoring agents are released and mixed with an aerosol generated by the aerosol emission device to change the flavor of the aerosol, so that the user can experience the aerosol with different flavors.

The flavor is added through the supporting element 500, since the flavor is coated in the flavor changing element, no flavor loss occurs during storage, and there is no problem of decomposition due to high temperature during atomization.

The supporting element 500 of the present embodiment can be made into a suitable geometric shape according to the inner space of the aerosol emission device, such as a cylindrical shape, a square column shape, or an elliptical column shape, etc. The axial supporting element through-hole 530 may not be provided, in this case, the flavor changing member may be pre-embedded in the supporting element when the supporting element is formed, or the supporting element may be punched radially for installing the flavor changing member.

### <Density of the Supporting Element>

The supporting element 500 of the present embodiment has a density of 0.08 g/cm³ to 0.35 g/cm³, such as 0.08 g/cm³, 0.10 g/cm³, 0.12 g/cm³, 0.15 g/cm³, 0.18 g/cm³, 0.21 g/cm³, 0.25 g/cm³, 0.3 g/cm³, 0.35 g/cm³, preferably 0.1 to 0.25 g/cm³. When the supporting element 500 has a density less than 0.08 g/cm³, it is difficult to manufacture the support element 500, and the strength of the support element 500 is insufficient, which is difficult to be assembled in the aerosol emission device; When the supporting element 500 has a density greater than 0.35 g/cm³, the strength of the support element 500 is too high, making it difficult to crack the flavor changing member by squeezing the support element during use.

Taking into account the convenience of manufacturing, assembly, and use, the present invention determines that the preferred density range for the support element 500 is 0.1 g/cm³ to 0.25 g/cm³, and the most preferred range is 0.12 g/cm³ to 0.2 g/cm³.

### <Bicomponent Fibers>

As shown in FIGS. 25a and 25b, the support element 500 according to the present embodiment has a three-dimensional network structure formed by thermally bonding bicomponent fibers 2, and the bicomponent fibers 2 have a sheath 21 and a core 22. The bicomponent fibers 2 may adopt bonding agent, plasticizer, or heat to bond fibers, preferably adopt heat to bind fibers to avoid the introduction of impurities during manufacturing the support element 500. The fiber components described in the present invention refer to the polymers that make fibers. Additives for the surface of the fibers, such as surfactants, are not considered to be the fiber components.

FIG. 25c shows an enlarged cross-sectional schematic view of bicomponent fibers of FIG. 25a and FIG. 25b. As shown in FIG. 25c, the sheath 21 and the core 22 are concentric structure. The bicomponent fibers 2 with the concentric structure have a greater rigidity, are easy to produce and are lower in price.

FIG. 25d shows another enlarged cross-sectional schematic view of the bicomponent fibers of FIG. 25a and FIG. 25b. As shown in FIG. 25d, the sheath 21 and the core 22 are eccentric structure. The bicomponent fibers 2 with an eccentric structure are relatively soft and fluffy, and it is easy to manufacture the support element 500 having a lower density. In addition, the support element 500 can be manufactured by using the bicomponent fibers with a coordinate structure, but thermal bonding is difficult. Of course, the support element 500 can be manufactured by using three-component sheath-and-core structural fibers, but the three-component sheath-and-core structural fibers are difficult to manufacture, have high cost and are poorer in cost performance.

The bicomponent fibers 2 are filaments or staple fibers. The support element 500 made from filaments has a higher strength, and the support element 500 made from short fibers has a better elasticity. The manufacturer can select suitable bicomponent fibers to make the support element 500 with a suitable density and suitable shape according to the performance requirements of the support element 500.

The core 22 of the bicomponent fibers 2 has a higher melting point than the sheath 21 by 25° C. or more. The support element 500 of the present embodiment is made by thermally bonding the bicomponent fibers 2 with a sheath-and-core structure, and the core 22 of the bicomponent fibers 2 has a higher melting point than the sheath 21 by 25° C. or more, so that the core 22 can maintain a certain rigidity when thermal bonding is performed between the fibers, which is convenient to manufacture the support element 500 having a lower density.

The sheath 21 of the bicomponent fibers 2 can be polyethylene, polypropylene and other polyolefins, or copolyester of ethylene terephthalate, polyamide-6, polylactic acid and other common polymer. The polyolefins are polymer of olefins, which are a general term for a class of thermoplastic resins usually obtained by independently polymerizing or copolymerizing α-olefins such as ethylene, propylene, 1-butene, 1-pentene, and 1-hexene and the like. The polyolefins have an inert molecular structure, which do not contain active groups on the molecular chain, and hardly adsorb flavor agents, so that it has unique advantages.

When the sheath 21 is polyethylene, such as linear low-density polyethylene, low-density polyethylene or high-density polyethylene, the core 22 may be polymer such as polypropylene, polyethylene terephthalate, etc. When the sheath 21 is polypropylene and polyolefin, the core 22 may be polyethylene terephthalate, polytrimethylene terephthalate or polybutylene terephthalate, polyamide, etc. The sheath 21 of the bicomponent fibers 2 has a low melting temperature, which is beneficial to improve the production efficiency and reduce the energy consumption in the manufacturing process.

When the sheath 21 is polylactic acid, according to the melting point of polylactic acid, if the sheath 21 adopts polylactic acid with a melting point of about 130° C., the core 22 may be polypropylene, polyethylene terephthalate, polylactic acid with a melting point of about 170° C. etc. When the sheath 21 is polylactic acid with a melting point of about 170° C., the core 22 may be polyethylene terephthalate, polybutylene terephthalate, polytrimethylene terephthalate, nylon, polyamide, etc. Polylactic acid is a biodegradable material, which can reduce environmental pollution caused by discarding the support element 500.

### <Fineness of Bicomponent Fibers>

The bicomponent fibers 2 from which the supporting element 500 of the present invention is made have a fineness of 1-30 denier, preferably 1-15 denier, most preferably 1.5-10 denier. The bicomponent fibers 2 with the sheath-and-core structure which has a fineness less than 1 denier is difficult to manufacture and has high cost. It is difficult to make the supporting element 500 from fibers with a fineness higher than 30 denier. The bicomponent fibers 2 which has the sheath-and-core structure and has 1-15 denier is easily thermally bonded into the supporting element 500 which has a lower density and has the three-dimensional structure with a suitable capillary force, and the bicomponent fibers 2 with the sheath-and-core structure having 1.5-10 denier is particularly suitable and has lower cost.

In the present embodiment, the bicomponent fibers 2 preferably have a fineness of 1.5 denier, 2 denier, 3 denier or 6 denier, the sheath 21 is polyethylene having a melting point of about 130° C., the core 22 is polypropylene having a melting point of about 165° C. and the supporting element 500 has a density of 0.1 to 0.25 g/cm³.

Although the supporting element 500 may also be made from monocomponent fibers. such as polypropylene fibers, by bonding with bonding agent, the use of the bonding agent often generally makes it difficult for the supporting element 500 to conform related regulations of food or drugs, so that the supporting element 500 is not suitable for use in aerosol emission devices such as electronic cigarettes, drug atomization, etc.

As shown in FIGS. 25a, 25b, 25c and 25d, in the present embodiment, the supporting element 500 has the three-dimensional network structure formed by thermally bonding the bicomponent fibers 2 with a concentric structure or an eccentric structure. The fineness of bicomponent fibers 2 is 3 denier, the outer layer 21 is polyethylene with a melting point of about 130° C. and the core 22 is polyethylene terephthalate with a melting point of about 270° C. The support element 500 made of it has a density of 0.1 g/cm³ to 0.25 g/cm³. The shape of the supporting element 500 is a cylinder, the outer diameter is 7.5 mm, and the supporting element 500 is provided with a supporting element through-hole 530 which is an axial through-hole having a diameter of 3.5 mm. The shape and size of the supporting element 500 is suitable for use in an emulated cigarette-shaped electronic cigarette. The outer diameter of the cross-sectional area of the support element 500 and the size of the supporting element through-hole 530 along the axial direction can be changed to produce different sizes of the support element 500, which are convenient for use in different aerosol emission devices. In the present embodiment, the sheath 21 of the bicomponent fibers 2 can be replaced by polylactic acid having a melting point of about 130° C., and the prepared supporting element 500 has similar properties.

When the aerosol generated by the atomization of the aerosol emission device 900 flows through the supporting element 500, the condensate generated during cooling the aerosol can be partially absorbed by the supporting element 500, thereby reducing the condensate in the aerosol and improving the consumption experience.

### The Twenty-Sixth Embodiment

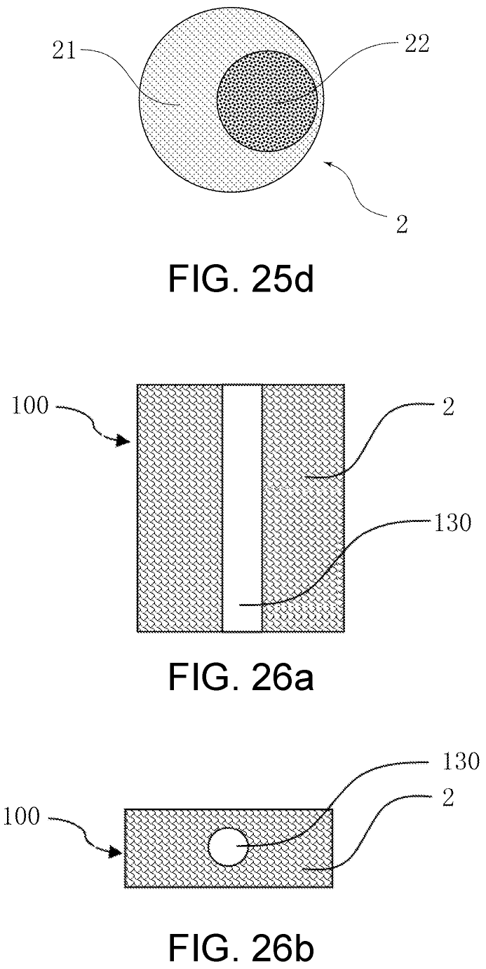

FIG. 26a shows a longitudinal section view of a supporting element according to the twenty-sixth embodiment of the present invention. FIG. 26b shows a cross-sectional view of the supporting element according to the twenty-sixth embodiment of the present invention. The structure of the present embodiment is similar to that of the twenty-fifth embodiment, and the same parts as the twenty-fifth embodiment will not be repeated in the description of this embodiment.

As shown in FIGS. 26a and 26b, in the present embodiment, a supporting element 500 has a three-dimensional network structure formed by thermally bonding bicomponent filaments with a concentric structure. The bicomponent fibers 2 have a fineness of 6 denier, and the sheath 21 is polypropylene having a melting point of about 165° C. and the core 22 is polybutylene terephthalate having a melting point of about 230° C. the supporting element 500 has a higher temperature resistance, and the prepared supporting element 500 has a density of 0.25 to 0.35 g/cm³. which has a greater rigidity, and is suitable for high-speed automated assembly. The cross-sectional view of the supporting element 500 shows a cuboid, the supporting element 500 is provided with a supporting element through-hole 530 which is an axial through-hole having a diameter of 3 mm, the shape of the supporting element 500 is suitable for using in cuboid-shaped flat cigarettes. In the present embodiment, the sheath 21 of the bicomponent fibers 2 can be replaced by polylactic acid having a melting point of about 170° C. and the prepared supporting element 500 has similar properties.

### The Twenty-Seventh Embodiment

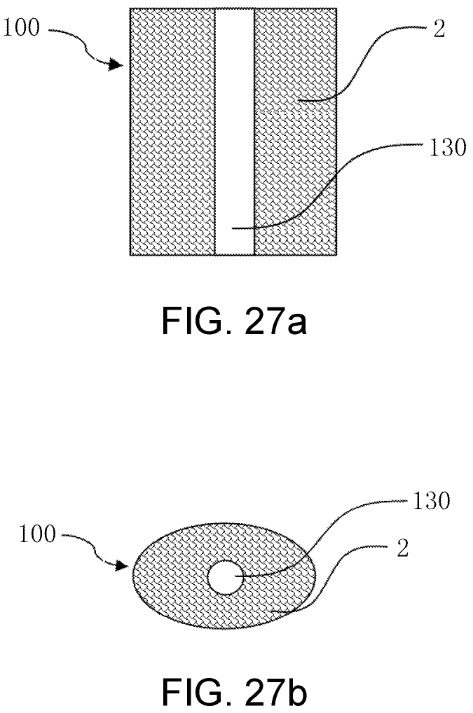

FIG. 27a shows a longitudinal section view of a supporting element 500 according to the twenty-seventh embodiment of the present invention. FIG. 27b shows a cross-sectional view of the supporting element 500 according to the twenty-seventh embodiment of the present invention. The structure of the present embodiment is similar to that of the twenty-fifth embodiment, and the same parts as the twenty-fifth embodiment will not be repeated in the description of this embodiment.

As shown in FIGS. 27a and 27b, in the present embodiment, a supporting element 500 has a three-dimensional network structure formed by thermally bonding bicomponent fibers 2 with an eccentric structure, and the bicomponent fibers 2 are staple fibers and have a fineness of 2 denier, the sheath 21 is polylactic acid having a melting point of 130° C. the core 22 is polylactic acid having a melting point of 155-185° C. and the prepared supporting element 500 has a density of 0.12 to 0.2 g/cm³. The cross-sectional view of the supporting element 500 shows an oval shape, the supporting element 500 is provided with an axial supporting element through-hole 530 having a diameter of 2.5 mm, and its shape is suitable for using in elliptical column-shaped flat cigarettes. In the present embodiment, the supporting element 500 is completely made of polylactic acid, which can be completely biodegraded and has a great significance for reducing environmental pollution.

In summary, the present invention involves the supporting element 500 that is used for the aerosol emission device and adopts the bicomponent fibers with the sheath-and-core structure, and the supporting element 500 can be made into a required size and shape of the three-dimensional structure in a thermally bonding process according to the application requirements to be suitable for high-speed automated assembly, so as to reduce the manufacturing cost of the aerosol emission device. The foregoing embodiments of the present invention are only intended to illustrate the principle and advantages of the present invention rather than limiting the present invention. For example, the supporting element 500 may be made by mixing two kinds of bicomponent fibers having different deniers, or some monocomponent fibers are mixed with the bicomponent fibers in order to reduce the cost without affecting the overall performance of the supporting element 500. Those skilled in the art can make modifications or changes to the foregoing embodiments without departing from the spirit and scope of the present invention. Therefore, all equivalent modifications or changes made by those skilled in the art without departing from the spirit and technical concepts disclosed by the present invention shall still be covered by the claims of the present invention.

In the claims:

1. A liquid storage element for storing and releasing liquids in an aerosol emission device, wherein the liquid storage element has a three-dimensional network structure formed by thermally bonding bicomponent fibers, the bicomponent fibers have a sheath and a core.

2. The liquid storage element according to claim 1, wherein the liquid storage element has a liquid storage element through-hole axially penetrating through the liquid storage element.

3. The liquid storage element according to claim 1, wherein the liquid storage element has a density of 0.03 g/cm³ to 0.25 g/cm³.

4. The liquid storage element according to claim 3, wherein the liquid storage element has a density of 0.04 g/cm³ to 0.12 g/cm³.

5. The liquid storage element according to claim 1, wherein the sheath and the core are a concentric structure or an eccentric structure.

6. The liquid storage element according to claim 1, wherein the bicomponent fibers are filaments or staple fibers.

7. The liquid storage element according to claim 1, wherein the core of the bicomponent fibers has a higher melting point than the sheath by 25° C. or more.

8. The liquid storage element according to claim 1, wherein the sheath is polyolefin, copolyester of polyethylene terephthalate or polyamide-6.

9. The liquid storage element according to claim 7, wherein the sheath is polylactic acid.

10. The liquid storage element according to claim 9, wherein the core is polylactic acid.

11. The liquid storage element according to claim 1, wherein the bicomponent fibers have a fineness of 1 to 30 denier.

12. The liquid storage element according to claim 11, wherein the bicomponent fibers have a fineness of 1 to 15 denier.

13. The liquid storage element according to claim 1, wherein the liquid storage element has a low-density portion, a high-density portion, and a density increasing portion provided between the low-density portion and the high-density portion.

14. The liquid storage element according to claim 1, wherein the liquid storage element comprises a liquid storage portion and a liquid collection portion with an upper and lower structure, and the liquid collection portion has a higher density than the liquid storage portion.

15. The liquid storage element according to claim 1, wherein the liquid storage element comprises a liquid collection portion and a liquid storage portion which is coated on the outer peripheral wall of the liquid collection portion and has a lower density than the liquid collection portion.

16-64. (canceled)
